(19) United States
(12) Patent Application Publication
(10) Pub. No.: US 2026/0315269 A1
(43) Pub. Date: Oct. 15, 2026

(54) PORTABLE CONTAINER FOR CLEANING A MENSTRUAL COLLECTION DEVICE

(71) Applicant: Blean, LLC, Raleigh, NC (US)

(72) Inventors: Paula Andrea Leon Delgadillo, Raleigh, NC (US); Cape Chappell, Raleigh, NC (US)

(73) Assignee: Blean, LLC, Raleigh, NC (US)

(21) Appl. No.: 19/641,112

(22) Filed: Apr. 7, 2026

Related U.S. Application Data
(60) Provisional application No. 63/786,585, filed on Apr. 10, 2025.

(51) Int. Cl. A61F 5/455; A61L 2/10; A61L 103/15

(52) U.S. Cl. CPC A61F 5/4553; A61L 2/10; A61L 2103/15; A61L 2202/121; A61L 2202/122; A61L 2202/17

(57) ABSTRACT

A portable container to clean a menstrual collection device. The container includes flexible walls, wherein the container is operable to expand and collapse in a telescopic manner. As the container is operable to reduce its footprint to occupy a smaller space when not in use, portability and convenience are positively impacted. Further, the container includes a dividing mechanism to save extra water within a reservoir, thus allowing the user an extra wash cycle if needed. In one embodiment, the container includes a plurality of drainage ports, wherein each drainage port is located at a separate end of the container.

### CROSS REFERENCES TO RELATED APPLICATIONS

This application is related to and claims priority from the following U.S. patents and patent applications. This application claims priority to and the benefit of U.S. Provisional Patent Application No. 63/786,585, filed Apr. 10, 2025, which is incorporated herein by reference in its entirety.

### BACKGROUND OF THE INVENTION

### 1. Field of the Invention

The present invention relates to feminine hygiene products, and more specifically to a portable container for cleaning menstrual collection devices, including but not limited to, menstrual cups or discs.

### 2. Description of the Prior Art

It is generally known in the prior art to provide containers for menstrual cups.

Prior art patent documents include the following:

US Patent Pub. No. 2023/0141755 for Menstrual Cup Case by inventors Daniels, et al., filed Nov. 7, 2022 and published May 11, 2023, is directed to a carrying and cleaning case for a menstrual cup. In particular, the carrying and cleaning case for a menstrual cup is configured with at least two, but preferably three, segments comprising of a cleaning segment and a drying segment. The exterior of the case is configured with finger grips to prevent slippage.

US Patent Pub. No. 2022/0031898 for Container for cleaning and sterilizing menstrual cups by inventors Lequay, et al., filed Mar. 18, 2021 and published Feb. 3, 2022, is directed to a container for cleaning and sterilizing a menstrual cup, including a vessel with an inner cavity made of elastic material having a bell-shaped portion for collecting menses and gripping means for insertion and removal during menstrual cycle; a lid closing an open-top, the vessel and the lid being made of materials suitable for exposure to microwave radiation, holding means for connecting the menstrual cup to the lid with the gripping means, wherein the lid is reversible for fitting on the open-top in two mounting positions, the first mounting position with the holding means directed towards the inner cavity such that, when attached to the lid, the menstrual cup projects at least partially into the inner cavity, and the second mounting position with the lid reversed and the holding means directed away from the inner cavity such that, when attached to the lid, the menstrual cup projects outwardly.

U.S. Pat. No. 12,053,556 for Sanitizing device by inventor Martz, filed May 5, 2022 and issued Aug. 6, 2024, is directed to a sanitizing device particularly suitable for sanitizing personal use items such as a menstrual cup. The sanitizing device includes top and bottom half members, top and bottom reflective members positioned within the top and bottom half members, respectively, and a planar quartz glass member to ensure that all interior and exterior surfaces of the menstrual cup are adequately exposed to UVC light to achieve sanitization.

US Patent Pub. No. 2011/0248040 for Collapsible container by inventor McGregor, filed Apr. 12, 2010 and published Oct. 13, 2011, is directed to a collapsible container which is movable from a collapsed position having a reduced volume for storage and transport and an expanded position having dimensional rigidity and securely holding the contents of the container. The collapsible container has a plurality of outer wall segments having a side wall with a first and second edge, the outer wall segments being movable from a collapsed position wherein the plurality of outer wall segments are stacked within each other such that the height of the collapsed container is similar to the height of the individual outer wall segments and an expanded position for use of the collapsible container. The outer wall segments are provided with releasable locking means whereby the outer wall segments may be locked one to another in the expanded position and being releasable to allow for the collapsible container to be moved to the collapsed position. The releasable locking means are a releasable locking latch means adjacent a first edge of each of the outer wall segments and a locking latch means receptacle adjacent a second edge of the outer wall segments, the releasable locking latch means of an outer wall segment cooperating with the releasable locking latch means receptacle of an adjacent outer wall segment to releasably lock the collapsible container in the expanded position, the releasable locking latch means of an outer wall segment being disengageable from the releasable locking latch means receptacle of the adjacent outer wall segment to allow the collapsible container to be moved to the collapsed position.

US Patent Pub. No. 2023/0159222 for Collapsible shape-retaining containers by inventor Tsui, filed Dec. 2, 2022 and published May 25, 2023, is directed to collapsible containers and a method of their manufacture. The collapsible containers have one or more collapsible wall sections and a stiff upper and lower tier. The wall sections have living hinges and three or more tiers between the hinges. A thermoplastic elastomer layer may join separately made portions of the container together. The containers may be made by molding and overmolding. The containers include inter alia bulk liquid containers, jugs, tubs, baskets, bottles, and food containers. The method of manufacturing includes placing a container component and a matching container body comprising a stiff first tier, a stiff second tier, and a collapsible wall section in a mold; assembling the container body with the container component to close one end of the container body; and overmolding a thermoplastic layer around the container body and the container component.

### SUMMARY OF THE INVENTION

The present invention relates to feminine hygiene products, and more specifically to a portable container for cleaning menstrual collection devices, including but not limited to, menstrual cups or discs.

It is an object of this invention to provide a portable container to clean menstrual collection devices in public settings.

In one embodiment, the present invention includes a container for cleaning a menstrual collection device, including a body including a superior body segment and an inferior body segment, a divider including a valve and a plurality of valve adapters, a lid, and a strap, wherein the superior body segment and the inferior body segment are connected via the divider, wherein the superior body segment is connected to the lid via a threaded ring, wherein the superior body segment and the inferior body segment comprise flexible walls, wherein the flexible walls are operable to reversely fold back, causing the container to collapse for storage, wherein the lid includes a drainage port, and wherein the lid is configured to cover the drainage port with a drainage cap, wherein the threaded ring includes a notch, operable to receive at least one loop of the strap, wherein the valve is configured to rotate independently from the plurality of valve adapters, and wherein the menstrual collection device is stored in the superior body segment during cleaning.

In another embodiment, the present invention includes a container for cleaning a menstrual collection device, including a body including a superior body segment and an inferior body segment, a divider including a valve and a plurality of valve adapters, and a lid, wherein the divider is configured to connect the superior body segment and the inferior body segment, wherein the lid is configured to connect to the superior body segment, wherein the body includes flexible walls, wherein the container is operable to expand and collapse telescopically, wherein the lid includes a drainage port and a drainage cap, wherein the valve is configured to rotate independently from the plurality of valve adapters, wherein the valve includes a handwheel encircling the valve, wherein the inferior body segment is operable to hold a reservoir of water, and wherein the superior body segment is configured to store the menstrual collection device.

In yet another embodiment, the present invention includes a container for cleaning a menstrual collection device, including a superior body segment, an inferior body segment, a valve, a superior valve adapter, an inferior valve adapter, and a lid, wherein the lid is connected to the superior body segment via a threaded ring, wherein the superior body segment is connected to the valve via the superior valve adapter, wherein the inferior body segment is connected to the valve via the inferior body adapter, wherein both the superior body segment and the inferior body segment include flexible walls, wherein a force applied to at least one end of the container causes the flexible walls to reversibly fold back to an inverted position, wherein the lid includes a drainage port, wherein the valve is configured to rotate independently from the superior valve adapter, wherein the inferior body segment is operable to hold a reservoir of water, and wherein the superior body segment is configured to contain the menstrual collection device during cleaning.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1A illustrates a front perspective view of a container according to one embodiment of the present invention.

FIG. 1B illustrates an orthogonal top view of a container according to one embodiment of the present invention.

FIG. 1C illustrates an orthogonal front view of a container according to one embodiment of the present invention.

FIG. 1D illustrates an orthogonal side view of a container according to one embodiment of the present invention.

FIG. 2 illustrates an exploded view of a container according to one embodiment of the present invention.

FIG. 3A illustrates a front perspective view of a collapsed container according to one embodiment of the present invention.

FIG. 3B illustrates an orthogonal top view of a collapsed container according to one embodiment of the present invention.

FIG. 3C illustrates an orthogonal front view of a collapsed container according to one embodiment of the present invention.

FIG. 3D illustrates an orthogonal side view of a collapsed container according to one embodiment of the present invention.

FIG. 4A illustrates a front perspective view of a lid according to one embodiment of the present invention.

FIG. 4B illustrates an orthogonal top view of a lid according to one embodiment of the present invention.

FIG. 4C illustrates an orthogonal front view of a lid according to one embodiment of the present invention.

FIG. 4D illustrates an orthogonal side view of a lid according to one embodiment of the present invention.

FIG. 5A illustrates a front perspective view of a lid according to another embodiment of the present invention.

FIG. 5B illustrates a bottom perspective view of a lid according to another embodiment of the present invention.

FIG. 5C illustrates an orthogonal top view of a lid according to another embodiment of the present invention.

FIG. 5D illustrates a sectional view of a lid according to another embodiment of the present invention.

FIG. 5E illustrates an orthogonal front view of a lid according to another embodiment of the present invention.

FIG. 5F illustrates an orthogonal side view of a lid according to another embodiment of the present invention.

FIG. 6A illustrates a front perspective view of a drainage cap according to one embodiment of the present invention.

FIG. 6B illustrates an orthogonal top view of a drainage cap according to one embodiment of the present invention.

FIG. 6C illustrates an orthogonal front view of a drainage cap according to one embodiment of the present invention.

FIG. 6D illustrates an orthogonal side view of a drainage cap according to one embodiment of the present invention.

FIG. 7A illustrates a front perspective view of a drainage cap according to another embodiment of the present invention.

FIG. 7B illustrates a bottom perspective view of a drainage cap according to another embodiment of the present invention.

FIG. 7C illustrates an orthogonal top view of a drainage cap according to another embodiment of the present invention.

FIG. 7D illustrates a sectional view of a drainage cap according to another embodiment of the present invention.

FIG. 7E illustrates an orthogonal front view of a drainage cap according to another embodiment of the present invention.

FIG. 7F illustrates an orthogonal side view of a drainage cap according to another embodiment of the present invention.

FIG. 8A illustrates a front perspective view of a first ring according to one embodiment of the present invention.

FIG. 8B illustrates an orthogonal top view of a first ring according to one embodiment of the present invention.

FIG. 8C illustrates an orthogonal front view of a first ring according to one embodiment of the present invention.

FIG. 9A illustrates a front perspective view of a second ring according to one embodiment of the present invention.

FIG. 9B illustrates an orthogonal top view of a second ring according to one embodiment of the present invention.

FIG. 9C illustrates an orthogonal front view of a second ring according to one embodiment of the present invention.

FIG. 10A illustrates a front perspective view of a third ring according to one embodiment of the present invention.

FIG. 10B illustrates an orthogonal top view of a third ring according to one embodiment of the present invention.

FIG. 10C illustrates an orthogonal front view of a third ring according to one embodiment of the present invention.

FIG. 11A illustrates a front perspective view of a first ring according to another embodiment of the present invention.

FIG. 11B illustrates a bottom perspective view of a first ring according to another embodiment of the present invention.

FIG. 11C illustrates an orthogonal top view of a first ring according to another embodiment of the present invention.

FIG. 11D illustrates a sectional view of a first ring according to another embodiment of the present invention.

FIG. 11E illustrates an orthogonal front view of a first ring according to another embodiment of the present invention.

FIG. 12A illustrates a front perspective view of a second ring according to another embodiment of the present invention.

FIG. 12B illustrates an orthogonal top view of a second ring according to another embodiment of the present invention.

FIG. 12C illustrates a sectional view of a second ring according to another embodiment of the present invention.

FIG. 12D illustrates an orthogonal front view of a second ring according to another embodiment of the present invention.

FIG. 13A illustrates a front perspective view of a lid, drainage cap, and ring assembly according to another embodiment of the present invention.

FIG. 13B illustrates a bottom perspective view of a lid, drainage cap, and ring assembly according to another embodiment of the present invention.

FIG. 13C illustrates an orthogonal top view of a lid, drainage cap, and ring assembly according to another embodiment of the present invention.

FIG. 13D illustrates a sectional view of a lid, drainage cap, and ring assembly according to another embodiment of the present invention.

FIG. 13E illustrates an orthogonal front view of a lid, drainage cap, and ring assembly according to another embodiment of the present invention.

FIG. 13F illustrates an orthogonal side view of a lid, drainage cap, and ring assembly according to another embodiment of the present invention.

FIG. 13G illustrates a perspective exploded view of a lid, drainage cap, and ring assembly according to another embodiment of the present invention.

FIG. 13H illustrates an exploded view of a lid, drainage cap, and ring assembly according to another embodiment of the present invention.

FIG. 14A illustrates a front perspective view of a superior body segment according to one embodiment of the present invention.

FIG. 14B illustrates an orthogonal top view of a superior body segment according to one embodiment of the present invention.

FIG. 14C illustrates an orthogonal front view of a superior body segment according to one embodiment of the present invention.

FIG. 14D illustrates an orthogonal side view of a superior body segment according to one embodiment of the present invention.

FIG. 14E illustrates a sectional view of a superior body segment according to one embodiment of the present invention.

FIG. 15A illustrates a front perspective view of an inferior body segment according to one embodiment of the present invention.

FIG. 15B illustrates an orthogonal top view of an inferior body segment according to one embodiment of the present invention.

FIG. 15C illustrates an orthogonal front view of an inferior body segment according to one embodiment of the present invention.

FIG. 15D illustrates an orthogonal side view of an inferior body segment according to one embodiment of the present invention.

FIG. 16A illustrates a top perspective view of a superior valve adapter according to one embodiment of the present invention.

FIG. 16B illustrates a bottom perspective view of a superior valve adapter according to one embodiment of the present invention.

FIG. 16C illustrates an orthogonal top view of a superior valve adapter according to one embodiment of the present invention.

FIG. 16D illustrates an orthogonal front view of a superior valve adapter according to one embodiment of the present invention.

FIG. 16E illustrates an orthogonal side view of a superior valve adapter according to one embodiment of the present invention.

FIG. 17A illustrates a top perspective view of an inferior valve adapter according to one embodiment of the present invention.

FIG. 17B illustrates a bottom perspective view of an inferior valve adapter according to one embodiment of the present invention.

FIG. 17C illustrates an orthogonal top view of an inferior valve adapter according to one embodiment of the present invention.

FIG. 17D illustrates an orthogonal front view of an inferior valve adapter according to one embodiment of the present invention.

FIG. 18A illustrates a top perspective view of a superior valve adapter according to another embodiment of the present invention.

FIG. 18B illustrates a bottom perspective view of a superior valve adapter according to another embodiment of the present invention.

FIG. 18C illustrates an orthogonal bottom view of a superior valve adapter according to another embodiment of the present invention.

FIG. 18D illustrates a sectional view of a superior valve adapter according to another embodiment of the present invention.

FIG. 18E illustrates an orthogonal front view of a superior valve adapter according to another embodiment of the present invention.

FIG. 18F illustrates an orthogonal side view of a superior valve adapter according to another embodiment of the present invention.

FIG. 19A illustrates a front perspective view of an inferior valve adapter according to another embodiment of the present invention.

FIG. 19B illustrates an orthogonal top view of an inferior valve adapter according to another embodiment of the present invention.

FIG. 19C illustrates a sectional view of an inferior valve adapter according to another embodiment of the present invention.

FIG. 19D illustrates an orthogonal front view of an inferior valve adapter according to another embodiment of the present invention.

FIG. 19E illustrates an orthogonal side view of an inferior valve adapter according to another embodiment of the present invention.

FIG. 20A illustrates a front perspective view of a valve according to one embodiment of the present invention.

FIG. 20B illustrates an orthogonal top view of a valve according to one embodiment of the present invention.

FIG. 20C illustrates an orthogonal front view of a valve according to one embodiment of the present invention.

FIG. 20D illustrates an orthogonal side view of a valve according to one embodiment of the present invention.

FIG. 21A illustrates a front perspective view of a valve according to another embodiment of the present invention.

FIG. 21B illustrates an orthogonal top view of a valve according to another embodiment of the present invention.

FIG. 21C illustrates a sectional view of a valve according to another embodiment of the present invention.

FIG. 21D illustrates an orthogonal front view of a valve according to another embodiment of the present invention.

FIG. 21E illustrates an orthogonal back view of a valve according to another embodiment of the present invention.

FIG. 22A illustrates a top perspective view of a valve according to yet another embodiment of the present invention.

FIG. 22B illustrates a bottom perspective view of a valve according to yet another embodiment of the present invention.

FIG. 22C illustrates an orthogonal bottom view of a valve according to yet another embodiment of the present invention.

FIG. 22D illustrates a sectional view of a valve according to yet another embodiment of the present invention.

FIG. 22E illustrates an orthogonal front view of a valve according to yet another embodiment of the present invention.

FIG. 22F illustrates an orthogonal back view of a valve according to yet another embodiment of the present invention.

FIG. 23A illustrates a front perspective view of a divider and rings assembly according to another embodiment of the present invention.

FIG. 23B illustrates an orthogonal bottom view of a divider and rings assembly according to another embodiment of the present invention.

FIG. 23C illustrates a sectional view of a divider and rings assembly according to another embodiment of the present invention.

FIG. 23D illustrates an orthogonal front view of a divider and rings assembly according to another embodiment of the present invention.

FIG. 23E illustrates an orthogonal back view of a divider and rings assembly according to another embodiment of the present invention.

FIG. 23F illustrates a perspective exploded view of a divider and rings assembly according to another embodiment of the present invention.

FIG. 23G illustrates an exploded view of a divider and rings assembly according to another embodiment of the present invention.

### DETAILED DESCRIPTION

The present invention is generally directed to feminine hygiene products, and more specifically to a portable container for cleaning menstrual collection devices, including but not limited to, menstrual cups or discs.

In one embodiment, the present invention includes a container for cleaning a menstrual collection device, including a body including a superior body segment and an inferior body segment, a divider including a valve and a plurality of valve adapters, a lid, and a strap, wherein the superior body segment and the inferior body segment are connected via the divider, wherein the superior body segment is connected to the lid via a threaded ring, wherein the superior body segment and the inferior body segment comprise flexible walls, wherein the flexible walls are operable to reversely fold back, causing the container to collapse for storage, wherein the lid includes a drainage port, and wherein the lid is configured to cover the drainage port with a drainage cap, wherein the threaded ring includes a notch, operable to receive at least one loop of the strap, wherein the valve is configured to rotate independently from the plurality of valve adapters, and wherein the menstrual collection device is stored in the superior body segment during cleaning. In one embodiment, the drainage port is configured to receive water, wherein the water is contained in the superior body segment for cleaning. In one embodiment, the inferior body segment is operable to hold a reservoir of water for a second cleaning cycle. In one embodiment, the valve includes a handle, wherein the handle includes one or more textured surfaces, wherein the one or more textured surfaces include markings indicating a rotation direction for opening and/or closing the valve. In one embodiment, the divider includes at least one seal or rubber gasket. In one embodiment, the container is operable to fold close in increments. In one embodiment, the container is self-standing when containing a menstrual collection device. In one embodiment, the body comprises silicone.

In another embodiment, the present invention includes a container for cleaning a menstrual collection device, including a body including a superior body segment and an inferior body segment, a divider including a valve and a plurality of valve adapters, and a lid, wherein the divider is configured to connect the superior body segment and the inferior body segment, wherein the lid is configured to connect to the superior body segment, wherein the body includes flexible walls, wherein the container is operable to expand and collapse telescopically, wherein the lid includes a drainage port and a drainage cap, wherein the valve is configured to rotate independently from the plurality of valve adapters, wherein the valve includes a handwheel encircling the valve, wherein the inferior body segment and/or the superior body segment is operable to hold a reservoir of water, and wherein the superior body segment is configured to store the menstrual collection device. In one embodiment, the drainage cap is removably attached to the lid. In one embodiment, the container is self-standing when the reservoir is filled with water. In one embodiment, the container is self-standing when the reservoir is partially filled with water. In one embodiment, the container further includes a strap operable to support the container when hung by the strap. In one embodiment, the container further includes an ultraviolet (UV) module configured to sanitize and/or disinfect the menstrual collection device. In one embodiment, the body comprises silicone.

In yet another embodiment, the present invention includes a container for cleaning a menstrual collection device, including a superior body segment, an inferior body segment, a valve, a superior valve adapter, an inferior valve adapter, and a lid, wherein the lid is connected to the superior body segment via a threaded ring, wherein the superior body segment is connected to the valve via the superior valve adapter, wherein the inferior body segment is connected to the valve via the inferior body adapter, wherein both the superior body segment and the inferior body segment include flexible walls, wherein a force applied to at least one end of the container causes the flexible walls to reversibly fold back to an inverted position, wherein the lid includes a drainage port, wherein the valve is configured to rotate independently from the superior valve adapter, wherein the inferior body segment is operable to hold a reservoir of water, and wherein the superior body segment is configured to contain the menstrual collection device during cleaning. In one embodiment, the container further includes a strap. In one embodiment, the superior valve adapter includes a plurality of occluding walls, wherein the plurality of occluding walls block fluid flow when the valve is turned to a first position and allows fluid flow when the valve is turned to a second position. In one embodiment, the drainage port is operable to be covered by a drainage cap. In one embodiment, the superior body segment and the inferior body segment comprise silicone.

None of the prior art discloses a telescopic, modular container to clean a menstrual cup or disc. Advantageously, the present invention as described herein offers an innovative two-step rinse feature where users do not have to leave the public restroom stall to correctly clean their cups or discs, ensuring a hygienic, safe, stress-free, and discreet experience. None of the prior art discloses a container with a rotating divider to partition extra water into a reservoir for use in a second cleaning cycle and/or drainage ports at both ends of the container to independently drain fluid at each end of the container. The water reservoir holds additional clean water for a second rinse to thoroughly clean the menstrual cup or disc. Further, the portability and compactness of the present invention advantageously allow users to take the device anywhere with ease, providing convenience in other challenging contexts such as traveling, backpacking, and exercising.

Every month, 25% of the global population experience the natural biological process of menstruation, lasting two to seven days. To effectively manage menstruation hygiene, many women typically resort to using disposable menstrual products, such as sanitary pads and tampons. This results in about 19 billion pads and tampons currently being thrown away every year. Recently, reusable menstrual collection devices (e.g., menstrual cups and discs) are becoming more popular options. Menstrual cups and discs are flexible devices that collect blood by being inserted into the vagina. They are more sustainable, reducing the amount of single-use plastics waste. Furthermore, these reusable devices are typically made from medical-grade silicone, which is safer for the body. Recent studies have found lead and arsenic in tampons; additional information can be found in “Tampons as a source of exposure to metal(loid)s,” J. A. Shearston et al., published in Environment International Volume 190, (2024), which is incorporated herein by reference. Additionally, they are reusable for years (e.g., cups can last up to four to six years); thus, as a result, they are more budget-friendly to the consumer in the long run.

When cleaning reusable menstrual cups or discs, users need to wash their hands, remove the cup or disc, toss the blood in the toilet, wash it in the sink, and reinsert it. To safely perform this activity, individuals require a private area with access to water and clean surfaces. In one customer discovery study involving the present invention, 90% of participants were identified to perceive menstrual cups or discs as difficult to clean in places that are outside the comfort of their homes. Common reasons cited by participants include more time involved to clean the cup or disc, increased stress, and other pain points such as lack of hygiene, safety, resources, and privacy. For example, in a public restroom, users are unable to wash their hands after going into a stall. In the study, participants reported having to use toilet paper, wipes, water bottles, and portable bidets for cleaning in public, making the activity unsanitary, uncomfortable, and undesirable. Additionally, when emptying the cup, users may accidentally spill blood, resulting in discomfort and embarrassment to clean their menses in a public setting. These factors result in discontinued use and can be a disincentive for new users eager to pursue sustainable menstrual care practices. Thus, there is a need for a portable cleaning container operable to clean a reusable menstrual collection device in one sitting, providing for an improved user experience. Moreover, addressing these challenges can tackle period poverty and contribute to the mitigation of stigmas and taboos about menstrual cups or discs, allowing individuals to feel confident and free during their menstrual cycle and promoting sustainable practices on a global scale.

In one embodiment, the present invention is directed to a container for a menstrual collection device. The container includes flexible walls, wherein the container is operable to expand and collapse in a telescopic manner. Compared to other cases known in the art, the container of the present invention is designed to easily collapse for storage in a purse or backpack. Additionally, the portable container is operable to store additional water in a reservoir for use in a second cleaning cycle. Further, the container is modular, providing for components to be easily cleaned or replaced. In one embodiment, the method of use of the invention recites steps comprising filling the container for cleaning a menstrual collection device, placing the menstrual collection device (e.g., menstrual cup or disc) containing blood inside the container, closing the container to secure the menstrual collection device, shaking the container to clean the menstrual collection device, and opening the drain of the container to empty the discarded fluid. In one embodiment, the method further includes opening a valve of the container to access reserve clean water from a reservoir, wherein the reserve clean water is used during a second rinse cycle. Advantageously, the drain allows for the cup or disc to stay secure in the present invention during the draining process.

The container is configured to connect to additional modules, such as a UV sterilization module as a non-limiting example. In one embodiment, the container includes a plurality of drainage ports, wherein a first drainage port is located at a first end of the container and the second drainage port is located at a second end of the container, wherein the plurality of drainage ports are operable to independently drain fluid and/or receive water.

While the present invention is directed to a portable container for cleaning a menstrual collection device, one of ordinary skill in the art will appreciate the container is operable to individually clean any fluid contacting device, including but not limited to pacifiers, nipple shields, and/or nursing cups.

Referring now to the drawings in general, the illustrations are for the purpose of describing one or more preferred embodiments of the invention and are not intended to limit the invention thereto.

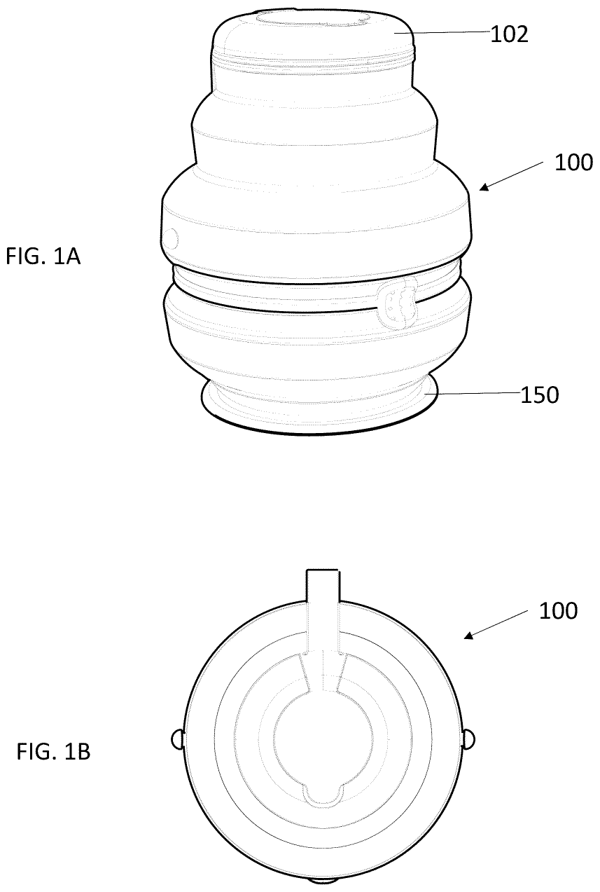

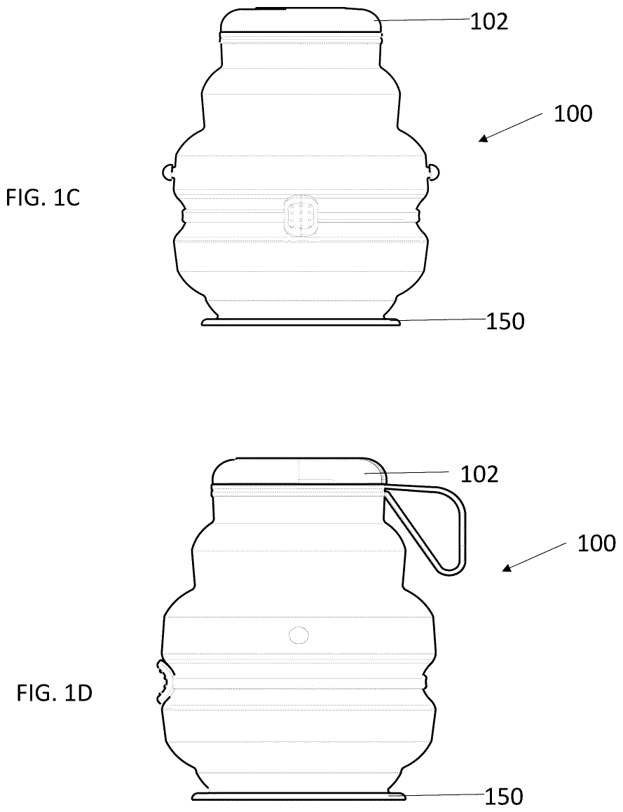

FIGS. 1A-D illustrates a container according to one embodiment of the present invention.

In one embodiment, the container 100 includes varying radii at different heights, wherein the external side surfaces of the container 100 include a plurality of ridges and indentations. In one embodiment, the expanded container 100 is bilaterally symmetrical. In one embodiment, the diameter of the base 150 is larger than the diameter of the lid 102, such that the container 100 is self-standing. In one embodiment, the container 100 is self-standing when substantially empty. In one embodiment, the container 100 is self-standing when containing a menstrual collection device. In one embodiment, the container 100 is self-standing when the reservoir is filled with water. In one embodiment, the container 100 is self-standing when the reservoir is partially filled with water.

In one embodiment, the height of the expanded container 100 is approximately 120 millimeters (mm). In one embodiment, the maximum width of the container 100 is approximately 97 mm. In one embodiment, the container 100 is operable to hold approximately 0.8 liters of water. In one embodiment, the container 100 is operable to hold between about 0.4 to about 0.8 liters of water. In one embodiment, the container 100 is operable to hold up to approximately a liter of water. In one embodiment, the container 100 is operable to hold between about 0.4 to about 0.8 liters of fluid, wherein the fluid includes water and blood.

In one embodiment, one or more components of the container 100 are formed from additive manufacturing, including but not limited to fused deposition modeling (FDM), stereolithography (SLA), and digital light processing (DLP). Alternatively, one or more components of the container 100 are formed from a different manufacturing process, including but not limited to injection molding, casting, computer numerical control (CNC) machining, or a combination. In one embodiment, the container 100 is formed from a combination of additive manufacturing techniques and subtractive manufacturing techniques. In one embodiment, the container 100 is not formed by manufacturing processes including overmolding or similar processes of combining materials. This along with the modular design of the container 100 results in parts operable to be easily refurbished.

In one embodiment, the material used for the container 100 is formed via additive manufacturing, and includes but is not limited to silicone, acrylonitrile butadiene styrene (ABS), polylactic acid (PLA), polyethylene terephthalate, glycol-modified polyethylene terephthalate (PETG), nylon, and a combination of various blends. One of ordinary skill in the art will appreciate that suitable materials are selected and can be mixed depending on desired strength, flexibility, preferred manufacturing method, and/or intended use. Preferably, each component of the container 100 is formed out of one material. Advantageously, this design results in parts operable to be easily refurbished. However, in an alternative embodiment, at least one component of the container 100 is formed of a combination of materials.

In one embodiment, any component designed to flex, including the superior body segment and inferior body segment, comprises a flexible material. In one embodiment, the flexible material is biocompatible. In a preferred embodiment, a flexible component operable to be in contact with the menstrual collection device, including the superior body segment, comprises silicone. In a preferred embodiment, silicone is food-grade or medical-grade. In one embodiment, an interior surface of a flexible component is infused and/or coated with at least one antimicrobial, antibacterial, or antifungal substance. In one embodiment, the antimicrobial substance includes a metal, metal alloy, or metallic compound that includes one or more of copper, silver, zinc, or combinations thereof. One of ordinary skill in the art will appreciate selecting a material with properties operable to disrupt or inhibit microbial biofilm but safe for human use.

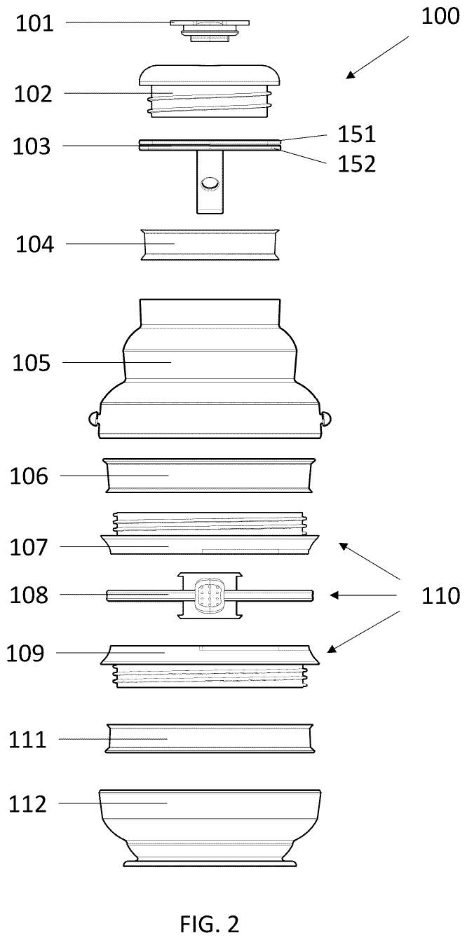

FIG. 2 illustrates an exploded view of a container according to one embodiment of the present invention. In one embodiment, the container 100 comprises a lid 102, a strap 103, a drainage cap 101, a plurality of rings 104, 106, 111, a superior body segment 105, an inferior body segment 112, and a divider 110. In one embodiment, the divider 110 includes a valve 108 and two valve adapters 107, 109. In a preferred embodiment, the divider 110 is a unitary assembly. In one embodiment, each component of the container 100 comprises an attachment means (e.g., threads) for connecting to an adjacent component. In one embodiment, the container 100 comprises a lid 102, a body including a superior body segment 105 and an inferior body segment 112, and a divider 110, wherein the lid 102 further includes a drainage cap 101. In one embodiment, the container 100 further comprises a strap 103. Given the valve adapters 107, 109 are configured to connect a valve 108 to the body segments 105, 112, one of ordinary skill in the art will understand a valve adapter is operable to be modified to include a plurality of rings 106, 111 to connect directly with the inner surface of the body segments 105, 112.

In one embodiment, the lid 102 is configured to connect to the superior body segment 105 via a first ring 104, wherein the lid 102 and the first ring 104 include threads. The lid 102 is operable to engage and disengage with the first ring 104. In one embodiment, the inner surface of the first ring 104 includes threads. In one embodiment, the superior body segment 105 is configured to receive the first ring 104. In one embodiment, the first ring 104 is permanently attached to the superior body segment 105. In one embodiment, the first ring 104 is securely attached to the superior body segment 105.

In one embodiment, the strap 103 includes two loops 151, 152, wherein a first loop 151 is operable to attach and detach with the lid 102, wherein the second loop 152 is operable to attach and detach with the superior body segment 105. In one embodiment, the second loop 152 is operable to attach and detach with the first ring 104. In one embodiment, the inner diameter of the first loop 151 is sized to fit around the lid 102, wherein when assembled, the first loop rests against the base of the top portion of the lid 102. In one embodiment, the inner diameter of the second loop 152 is sized to fit around the top portion of the superior body segment 105. In one embodiment, the inner diameter of the second loop 152 is sized to fit around the first ring 104. In one embodiment, the first ring 104 is rigid, thus provides reinforcement to the top portion of the superior body segment 105. This reinforcement provides a stable surface for the second loop 152 of the strap 103 to wrap around. In one embodiment, the loops 151, 152 are comprised of a flexible material. In one embodiment, the strap 103 is a unitary body. In one embodiment, the strap 103 is operable to be secured to the lid 102 and the superior body segment 105, wherein the strap 103 connects the lid 102 to the container 100. Securing the lid 102 to the superior body segment 105 allows the user to open the container 100 without misplacing the lid 102.

In one embodiment, the lid 102 is configured to receive a drainage cap 101. In one embodiment, the drainage cap 101 is removably attached to the lid 102 with a drainage cap strap. In one embodiment, the drainage cap 101 is permanently attached to the lid 102 with a drainage cap strap. In one embodiment, the lid 102 and drainage cap 101 are integrally formed. Securing the drainage cap 101 to the lid 102 allows the user to pop open the drainage cap without misplacing it.

In one embodiment, the superior body segment 105 is configured to connect to the divider 110 via a second ring 106, wherein the divider 110 and the second ring 106 include threads. The divider 110 is operable to engage and disengage with the second ring 106. In one embodiment, the inner surface of the second ring 106 includes threads. In one embodiment, the superior body segment 105 is configured to connect to the divider 110, wherein the divider 110 and second ring 106 form an integrally formed divider-and-connector assembly. In one embodiment, the second ring 106 is permanently attached to the superior body segment 105. In one embodiment, the second ring 106 is securely attached to the superior body segment 105.

In one embodiment, the valve 108 interfaces with a valve adapter 107, 109 by an interference fit. In a preferred embodiment, the valve 108 is configured to securely attach to the valve adapters 107, 109, forming a unitary assembly. In one embodiment, each of the valve adapters 107, 109 further include a plurality of joining pegs, and the valve 108 further includes a plurality of corresponding interior holes. One of ordinary skill in the art will appreciate the valve 108 and the valve adapters 107, 109 are operable to be joined together with suitable adhesive and/or other fasteners or fastening techniques so that a unitary assembly is formed. In an alternate embodiment, the valve 108 interfaces with a valve adapter 107, 109 by a transition fit. A transition fit provides for easy access to all disassembled components for cleaning and securely reassembling the components to maintain a watertight seal when in use.

In one embodiment, the inferior body segment 112 is configured to connect to the divider 110 via a third ring 111, wherein the divider 110 and the third ring 111 include threads. The divider 110 is operable to engage and disengage with the third ring 111. In one embodiment, the inner surface of the third ring 111 includes threads. In one embodiment, the inferior body segment 112 is configured to receive the third ring 111. In one embodiment, the inferior body segment 112 is configured to connect to the divider 110, wherein the divider 110 and third ring 111 form an integrally formed divider-and-connector assembly. In one embodiment, the third ring 111 is permanently attached to the inferior body segment 112. In one embodiment, the third ring 111 is securely attached to the inferior body segment 112.

In one embodiment, the assembled container 100 is watertight. One of ordinary skill in the art will appreciate the container is operable to include a gasket between connecting components such that the container remains watertight.

In one embodiment, the container 100 is operable to disassemble into components for easy cleaning. In one embodiment, the container 100 includes a UV module, wherein the UV module includes a UV light. In one embodiment, the UV module is operable to sanitize and/or disinfect the reusable menstrual collection device. In one embodiment, the UV module comprises UV shielding. In one embodiment, the container 100 is configured to receive the UV module via a plurality of adapters. In one embodiment, the UV module is substantially positioned in the middle of the container 100. In one embodiment, the UV module is configured to attach to the superior body segment 105. In one embodiment, the container 100 including the UV module does not include the divider 110. In one embodiment, the container 100 including the UV module does not include the divider 110 nor the inferior body segment 112. In one embodiment, the container 100 includes at least one additional module and a plurality of adapters, wherein the container 100 is configured to receive the additional module via the plurality of adapters.

In one embodiment, the container 100 includes a plurality of drainage ports and a plurality of drainage caps. The number of drainage caps is equal to the number of drainage ports. In one embodiment, each of the plurality of drainage ports is located at one end of the container 100, wherein each of the plurality of drainage ports is operable to drain fluid from the container 100. In one embodiment, at least one of the plurality of drainage caps is secured to the container 100 when opening the corresponding drainage port.

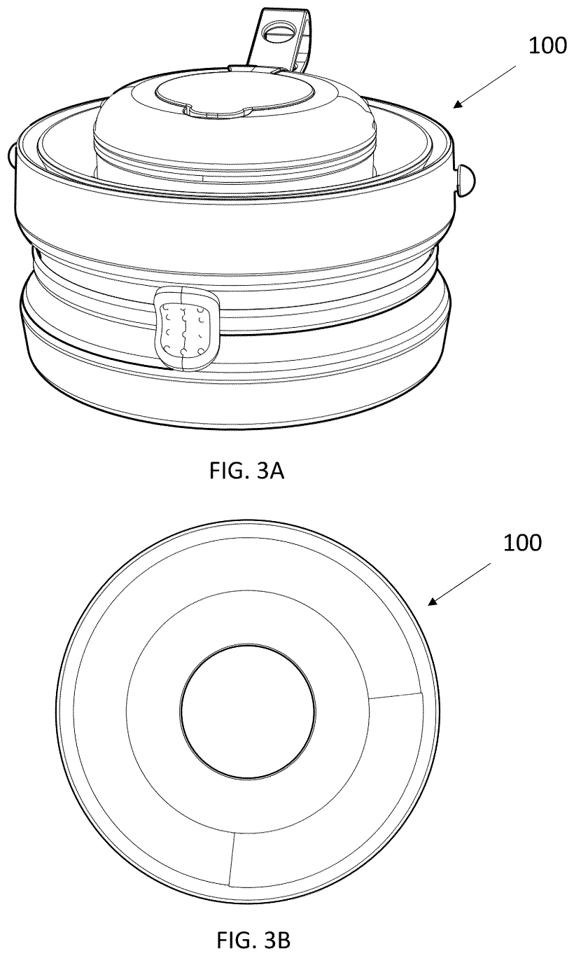

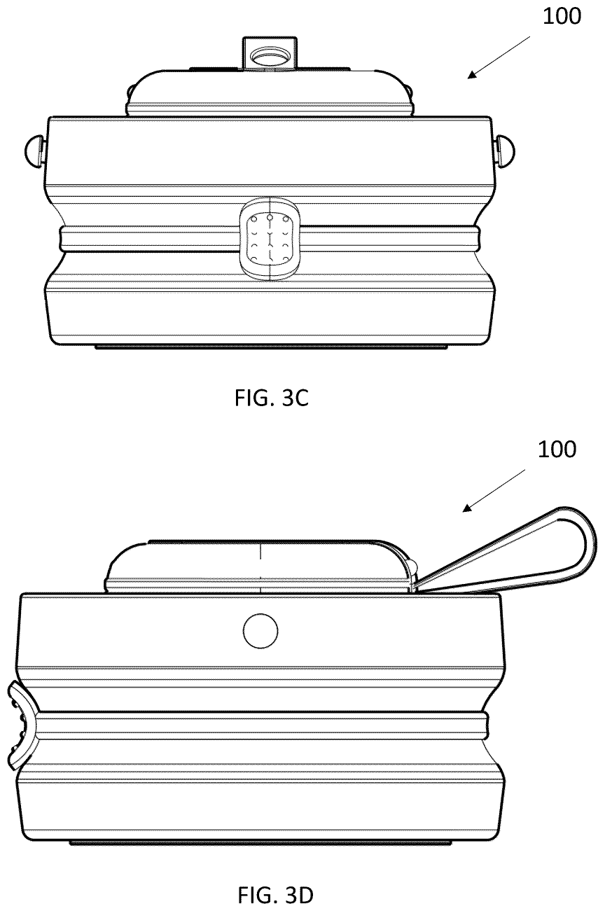

FIGS. 3A-D illustrates a perspective view of a container according to one embodiment of the present invention. In one embodiment, the height of the collapsed container 100 is approximately 60 mm. In one embodiment, the height of the collapsed container 100 is between about 40 mm to about 50 mm.

In one embodiment, a force applied to at least one end of the container 100 causes the flexible walls of the body segments to reversely fold back to an inverted position. In one embodiment, the container 100 includes crease lines between tiers of flexible walls, wherein when a force is applied to collapse the container 100, the flexible walls begin deforming at the crease lines.

In one embodiment, the container 100 is operable to fold close in increments. In one embodiment, when the container 100 is fully collapsed, the container 100 is operable to stand upright without external support. In one embodiment, to expand the collapsed container 100, a force is applied to move one end of the container 100 away from the other end. In one embodiment, the container 100 is operable to fold open in increments. In one embodiment, when the container 100 is fully expanded, the container 100 is operable to stand upright without external support.

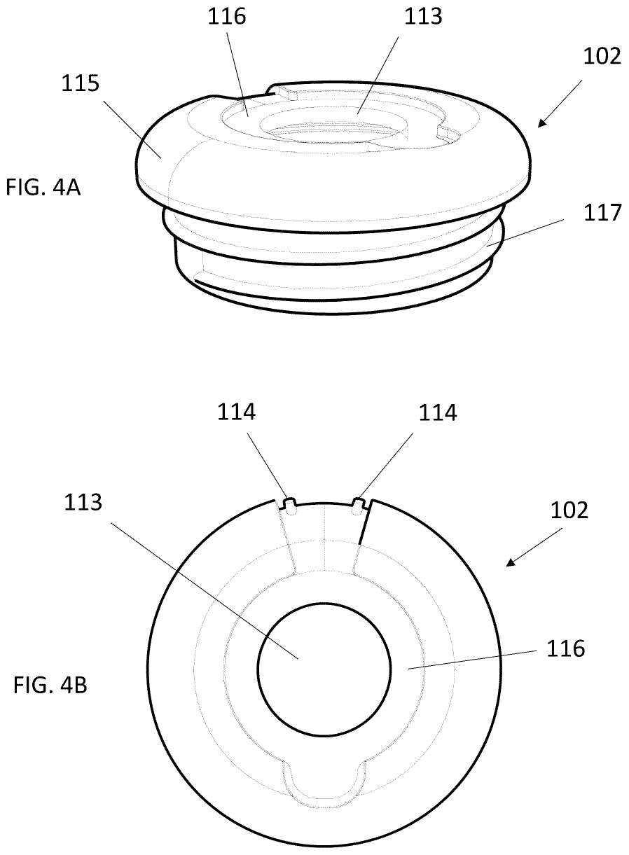

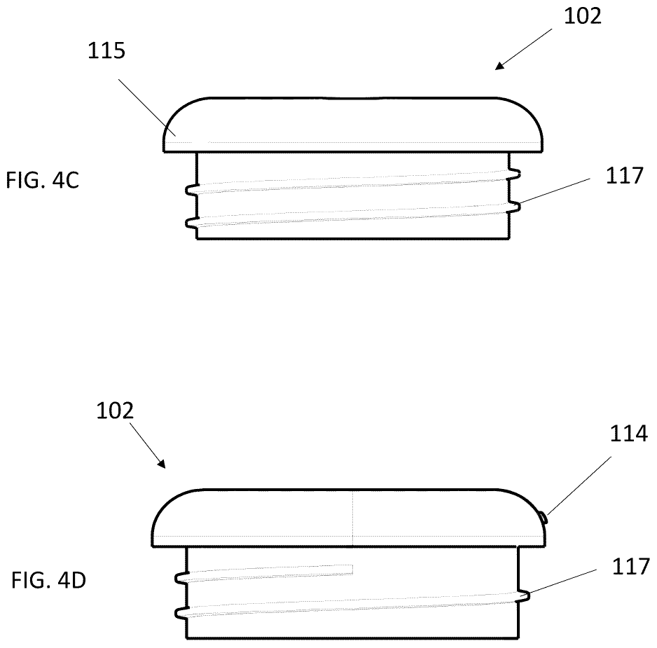

FIGS. 4A-D illustrate views of a lid according to one embodiment of the present invention. In one embodiment, the lid 102 includes a threaded post 117 and a top portion 115. In one embodiment, the maximum diameter of the lid 102 is approximately 60 mm. In one embodiment, the lid 102 includes a drainage port 113, wherein the drainage port 113 is operable to be covered by a drainage cap. In one embodiment, the diameter of the drainage port 113 is approximately 23 mm. In one embodiment, the lid 102 includes a plurality of posts 114, wherein the drainage cap strap is operable to be secured to the plurality of posts 114. In one embodiment, the lid 102 includes a post 114, wherein the drainage cap strap is operable to be secured to the post 114. One of ordinary skill in the art will appreciate selecting the location and number of posts to the attachment points on the drainage cap strap such that the drainage cap strap is securely fastened to the lid 102. In one embodiment, the drainage cap strap is operable to be removed from the lid 102. In one embodiment, the top portion 115 of the lid 102 includes a recessed surface 116 around the drainage port 113, wherein when the drainage cap is pressed into the drainage port 113, the drainage cap sits flush with the lid 102. In one embodiment, the height of the recess is about the thickness of the top portion of the drainage cap.

In an alternate embodiment, the lid 102 is attached to a first ring via a hinge. In yet another embodiment, the lid 102 is directly attached to the superior body segment via a hinge. When the lid 102 is attached via a hinge, the lid 102 does not include threads on the threaded post 117. In one embodiment, the container further includes a button for releasably locking the lid 102 closed. In one embodiment, pushing the button causes the lid to spring open.

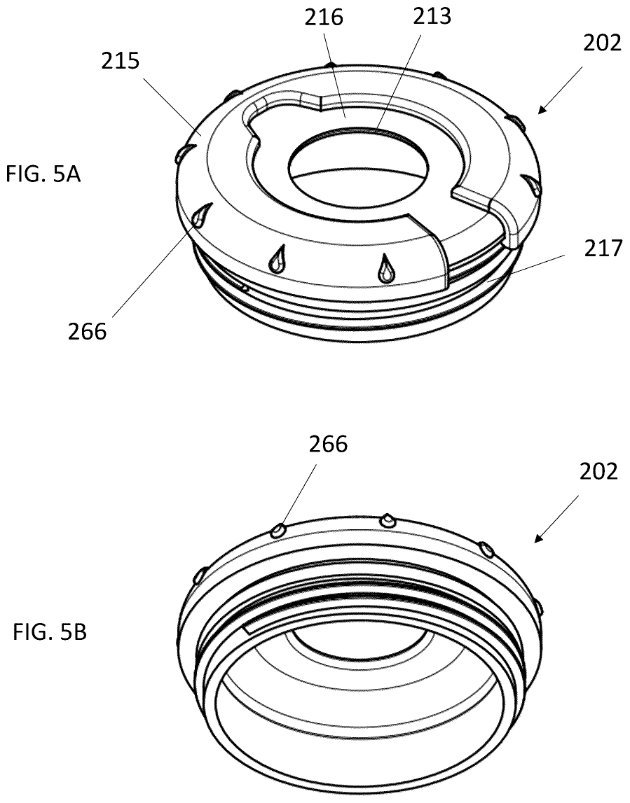

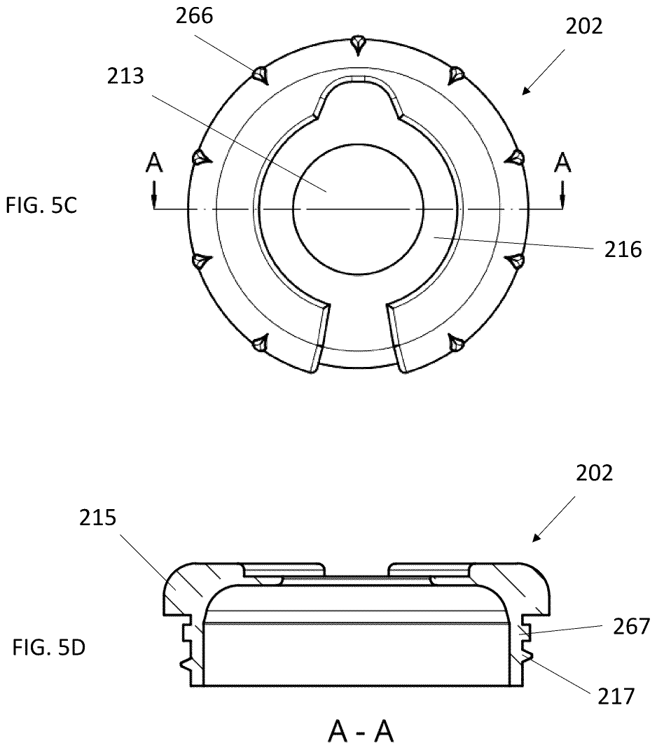

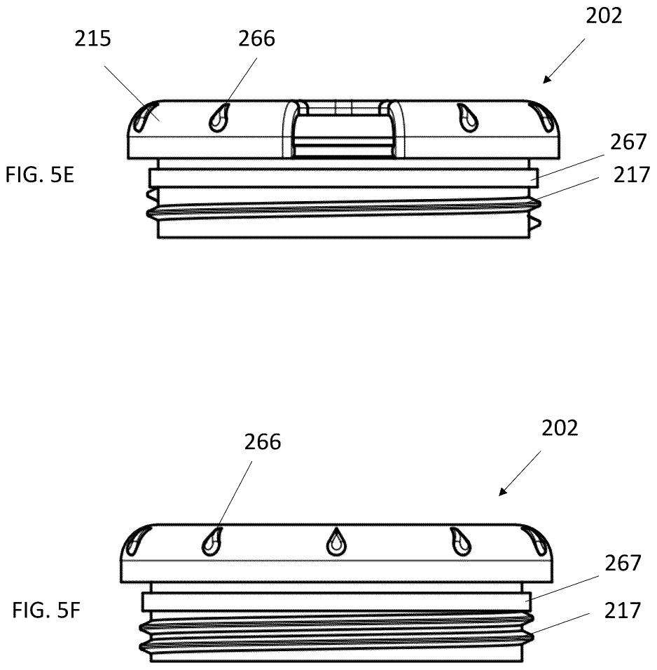

FIGS. 5A-F illustrate views of a lid according to another embodiment of the present invention. The lid 202 is substantially identical in structure and operation as the lid 102 of FIGS. 4A-D, except for the differences that will now be described. In one embodiment, the top surface 215 of the lid 202 includes a plurality of raised elements and/or divots 266. In one embodiment, the top surface 215 of the lid 202 is textured. The textured surface increases surface roughness to improve the user's grip when rotating the lid. In one embodiment, the plurality of raised elements and/or divots 266 are evenly spaced apart around the circumference of the lid 202. In one embodiment, the lid 202 includes a threaded post 217 and a top portion 215. In one embodiment, the maximum diameter of the lid 202 is approximately 60 mm. In one embodiment, the lid 202 includes a drainage port 213, wherein the drainage port 213 is operable to be covered by a drainage cap. In one embodiment, the diameter of the drainage port 213 is approximately 23 mm. In one embodiment, the drainage cap strap is operable to be removed from the lid 202. In one embodiment, the top portion 215 of the lid 202 includes a recessed surface 216 around the drainage port 213, wherein when the drainage cap is pressed into the drainage port 213, the drainage cap sits flush with the lid 202.

The lid 202 is operable to include a collar 267, wherein the collar provides a resting surface for the strap of the container to wrap around without slipping off the lid 202. In other words, at least one loop of the strap is operable to rest on top of the collar 267.

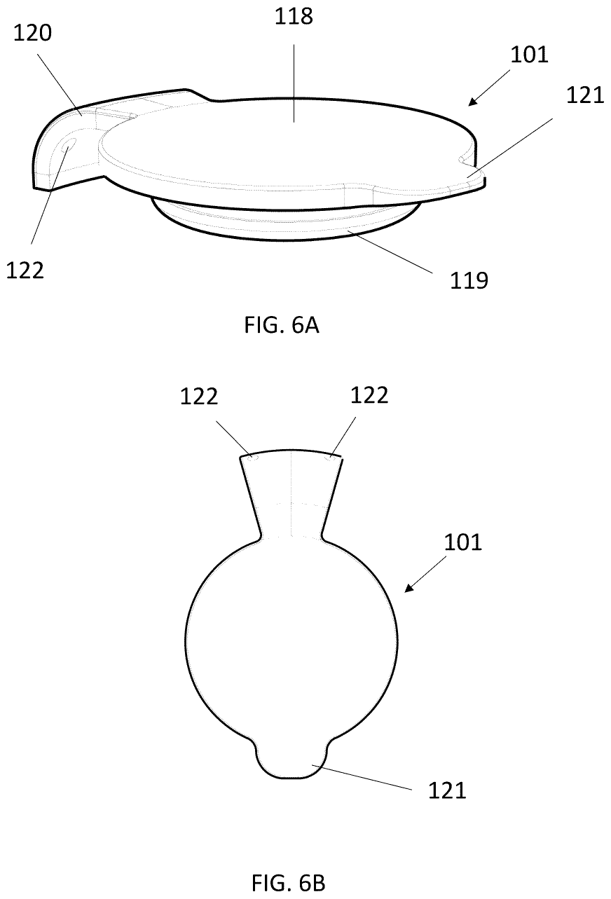

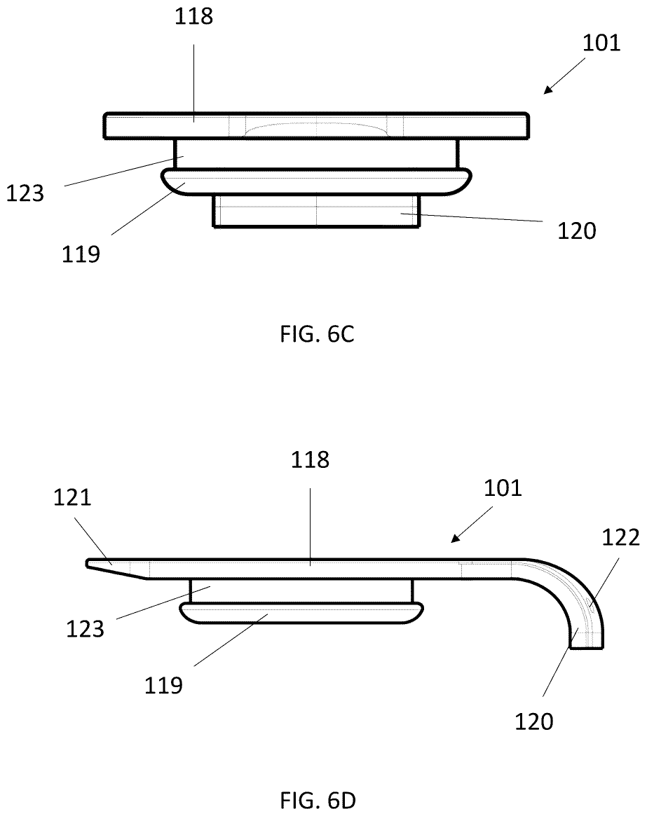

FIGS. 6A-D illustrate views of the drainage cap according to one embodiment of the present invention. In one embodiment, the drainage cap 101 comprises a top portion 118, a plug 119, and a drainage cap attachment strap 120. In one embodiment, the top portion 118 includes a tab 121. Advantageously, the tab 121 is designed for a user to easily pop the drainage cap 101 free from the drainage port. In one embodiment, when the drainage cap 101 is pushed into the lid, the tab 121 rests in the recessed surface of the lid.

In one embodiment, the drainage cap attachment strap 120 include a plurality of holes 122, wherein the plurality of holes 122 are configured to receive the plurality of posts of the lid, wherein the drainage cap attachment strap 120 is operable to be secured to the lid. In one embodiment, the drainage cap attachment strap 120 include a hole 122, wherein the hole 122 is operable to receive the post of the lid. The drainage cap attachment strap 120 is operable to secure the drainage cap to the lid when the drainage port is open. One of ordinary skill in the art will appreciate selecting the number and location of holes in the drainage cap attachment strap such that the drainage cap attachment strap is best secured to the lid.

In one embodiment, the plug 119 includes a spacer 123, wherein the plug 119 is sized to insert into the drainage port and create a watertight seal. In one embodiment, the total height of the plug 119 is approximately 4.5 mm. In one embodiment, the height of the spacer 123 is approximately 2.5 mm. In one embodiment, the diameter of the spacer 123 is sized to fit the drainage port.

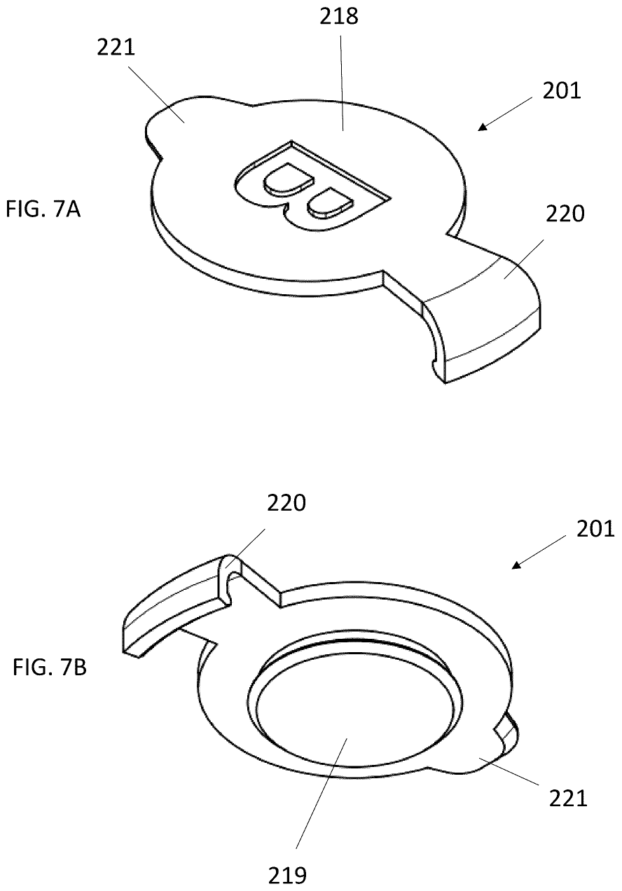

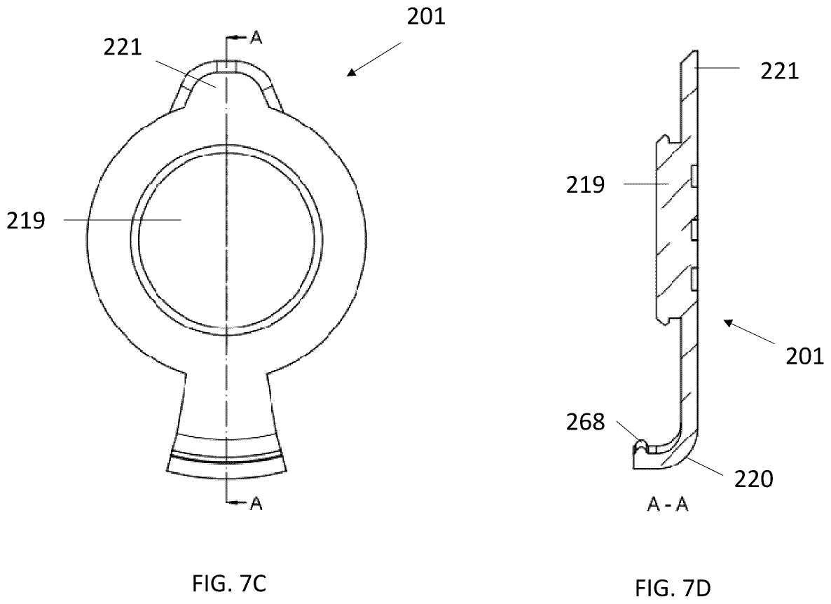

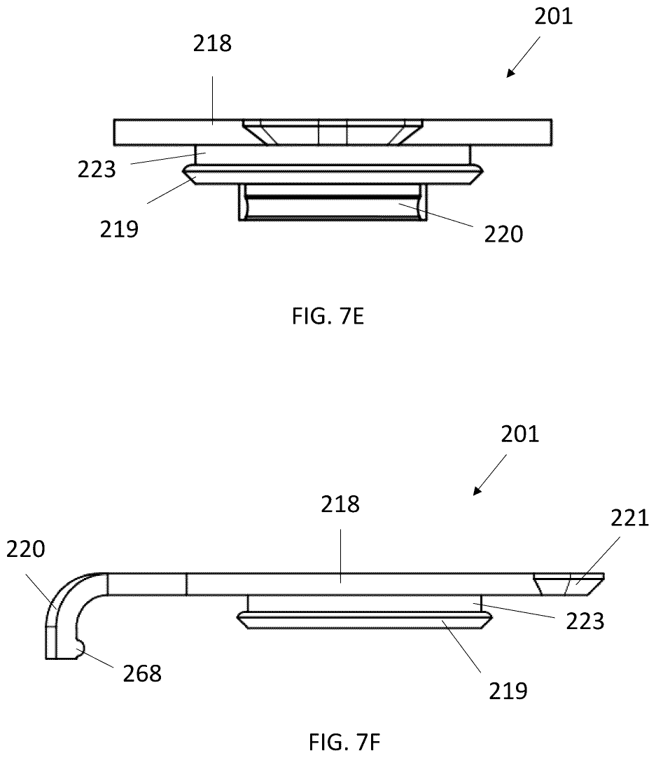

FIGS. 7A-F illustrate views of a drainage cap according to another embodiment of the present invention. The drainage cap 201 is substantially identical in structure and operation to the drainage cap 101 of FIGS. 6A-D, except for the differences described below. In one embodiment, the top surface 218 of the drainage cap 201 includes a logo or design. In one embodiment, the drainage cap attachment strap 220 includes a protrusion 268, wherein the lid includes a corresponding recess, such that the drainage cap 201 is secured to the lid when fitted together. Because the protrusion 268 and corresponding recess are operable to “lock” together, the drainage cap 201 is operable to substantially flex away from the drainage port of the lid while remaining secured to the lid. Thus, the user is operable to remove the drainage cap to access the drainage port without accidentally dropping or losing the drainage cap 201. In one embodiment, the drainage cap 201 comprises the top surface 218, a plug 219, and the drainage cap attachment strap 220. In one embodiment, the top surface 218 includes a tab 221. Advantageously, the tab 221 is designed for a user to easily disengage the drainage cap 201 free from the drainage port. In one embodiment, the plug 219 includes a spacer 223, wherein the plug 219 is sized to insert into the drainage port and create a watertight seal.

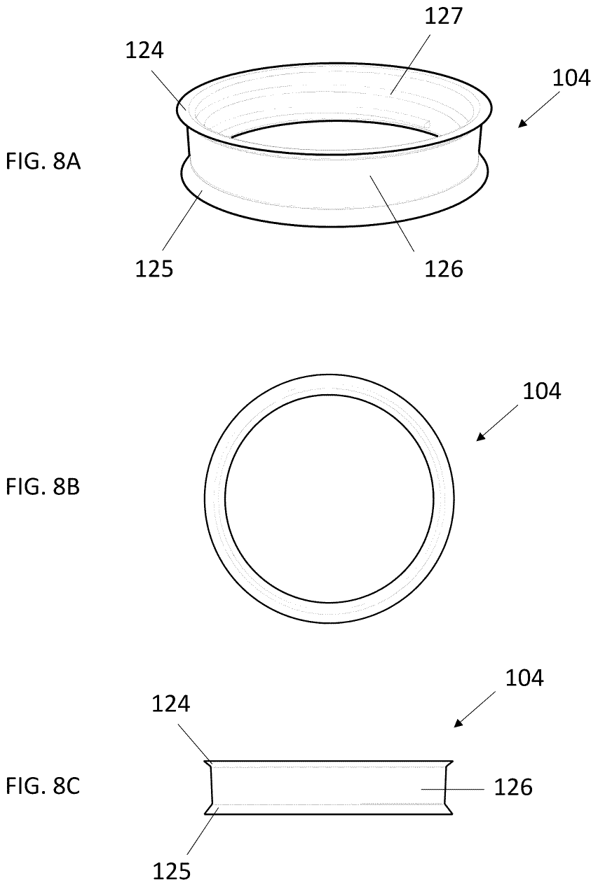

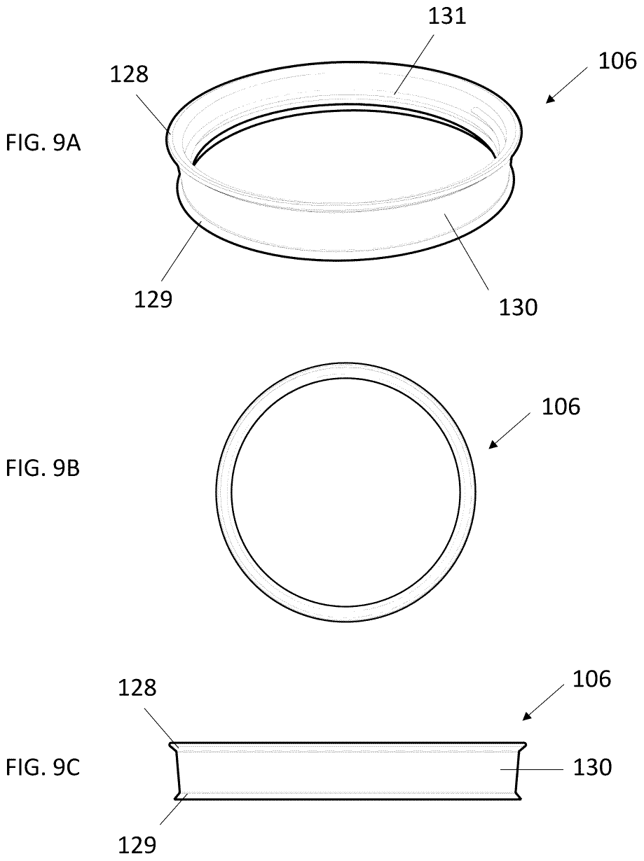

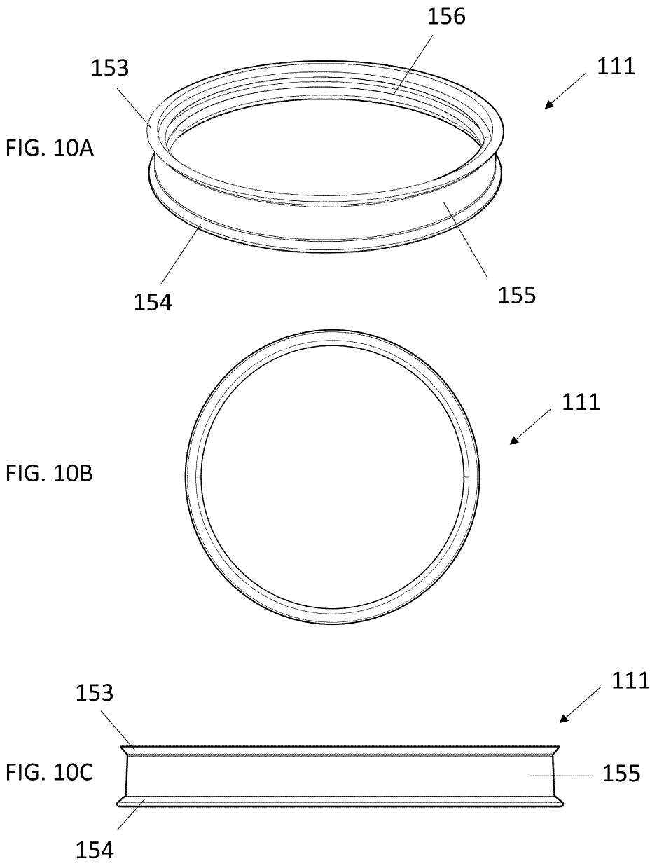

FIGS. 8A-10C illustrate views of a ring according to one embodiment of the present invention. While FIGS. 8A-C illustrate a first ring 104 according to one embodiment of the present invention, FIGS. 9A-C illustrate a second ring 106 according to one embodiment of the present invention, and FIGS. 10A-C illustrate a third ring 111 according to one embodiment of the present invention, one of ordinary skill in the art will understand these are not intended to be limiting and are applicable to any ring of the present invention, wherein the ring connects two components together. In one embodiment, the first ring 104 includes a central ring body 126 and two flanges 124, 125, wherein each flange 124, 125 is at one end of the ring. In one embodiment, the first ring 104 further includes a threaded interior ring body 127. In a preferred embodiment, the first ring 104 is a unitary body. Advantageously, the first ring 104 is designed to lock into place with the superior body segment. Similarly, the third ring 111 is designed to lock into place with the inferior body segment.

In one embodiment, the outer diameter of the top flange 124 is sized to match the diameter at the base of the top surface of the lid, resulting in an effective seal when the container is closed. In one embodiment, the outer diameter of the top flange 124 is approximately 60 mm. In one embodiment, the outer diameter of the bottom flange 125 is sized to fit inside the superior body segment. In one embodiment, the outer diameter of the bottom flange 125 is approximately 60 mm. In one embodiment, the outer diameter of the top flange 124 is substantially the same as the outer diameter of the bottom flange 125. In one embodiment, the outer diameter of the top flange 124 is smaller than the outer diameter of the bottom flange 125. In one embodiment, the outer diameter of the top flange 124 is larger than the outer diameter of the bottom flange 125. In one embodiment, when the superior body segment is assembled with the first ring 104, the flanges 124, 125 prevent the first ring 104 from moving out of position with the superior body segment. In one embodiment, the height of the first ring 104 is sized to fit inside the top portion of the superior body segment. In one embodiment, the height of the first ring 104 is approximately 13 mm. In one embodiment, the central ring body 126 is tapered. In a preferred embodiment, the threads of the threaded interior ring body 127 are parallel. In one embodiment, the threads are tapered. In one embodiment, the central ring body 126 is sized to fit inside the top portion of the superior body segment. In one embodiment, the average outer diameter of the central ring body 126 is approximately 56 mm. In one embodiment, the inner diameter of the central ring body 126 is sized to fit the threaded post of the lid. In one embodiment, the inner diameter of the central ring body 126 is approximately 50 mm.

In one embodiment, the outer diameter of the top flange 128 is sized to fit inside the superior body segment. In one embodiment, the outer diameter of the top flange 128 is approximately 92 mm. In one embodiment, the outer diameter of the bottom flange 129 is sized to match the maximum diameter at the collar of the superior valve adapter, resulting in an effective seal when the container is assembled. In one embodiment, the outer diameter of the bottom flange 129 is approximately 89 mm. In one embodiment, the outer diameter of the top flange 128 is substantially the same as the outer diameter of the bottom flange 129. In one embodiment, the outer diameter of the top flange 128 is smaller than the outer diameter of the bottom flange 129. In one embodiment, the outer diameter of the top flange 128 is larger than the outer diameter of the bottom flange 129. In one embodiment, when the superior body segment is assembled with the second ring 106, the flanges 128, 129 prevent the second ring 106 from moving out of position with the superior body segment. In one embodiment, the height of the second ring 106 is sized to fit inside the bottom of the superior body segment. In one embodiment, the height of the second ring 106 is approximately 15 mm. In one embodiment, the central ring body 130 is tapered. In a preferred embodiment, the threads of the threaded interior ring body 131 are parallel. In one embodiment, the threads are tapered. In one embodiment, the central ring body 130 is sized to fit inside the top portion of the superior body segment. In one embodiment, the average outer diameter of the central ring body 130 is approximately 85 mm. In one embodiment, the inner diameter of the central ring body 130 is sized to fit the threaded body of the superior valve adapter. In one embodiment, the inner diameter of the central ring body 126 is approximately 82 mm.

In one embodiment, the outer diameter of the top flange 153 of the third ring 111 is sized to match the diameter at the inferior valve adapter, resulting in an effective seal when the container is assembled. In one embodiment, the outer diameter of the bottom flange 154 of the third ring 111 is sized to fit the top portion of the inferior body segment. In one embodiment, the outer diameter of the top flange 153 of the third ring 111 is substantially the same as the outer diameter of the bottom flange 154 of the third ring 111. In one embodiment, the outer diameter of the top flange 153 of the third ring 111 is smaller than the outer diameter of the bottom flange 154 of the third ring 111. In one embodiment, the outer diameter of the top flange 153 of the third ring 111 is larger than the outer diameter of the bottom flange 154 of the third ring 111. In one embodiment, when the inferior body segment is assembled with the third ring 111, the flanges of the third ring 111 prevent the third ring 111 from moving out of position with the inferior body segment. In one embodiment, the height of the third ring 111 is sized to fit inside the top portion of the inferior body segment. In one embodiment, the central ring body 155 of the third ring 111 is tapered. In a preferred embodiment, the threads of the threaded interior ring body 156 are parallel. In one embodiment, the threads are tapered. In one embodiment, the central ring body 155 of the third ring 111 is sized to fit inside the top portion of the inferior body segment. In one embodiment, the inner diameter of the central ring body 155 of the third ring 111 is sized to fit the threaded body of the inferior valve adapter.

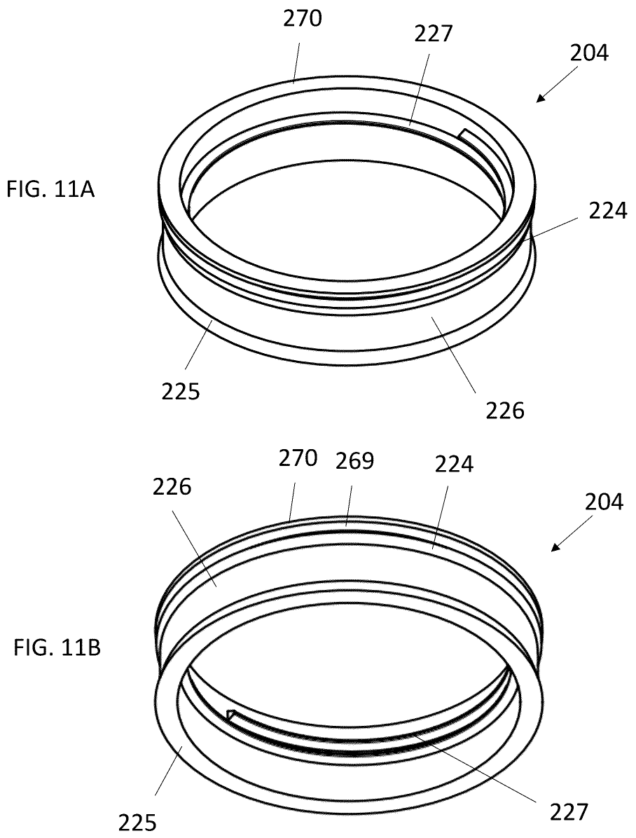

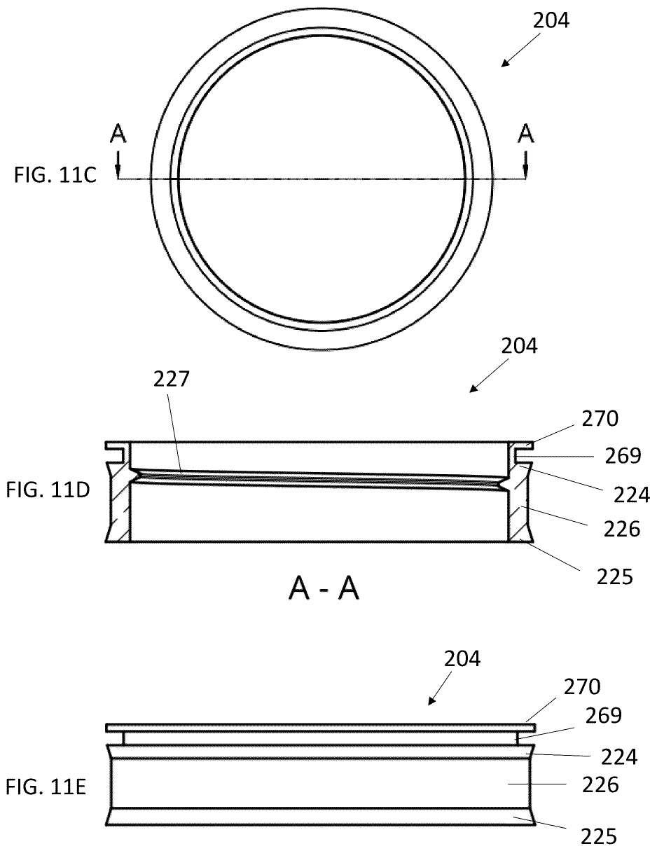

FIGS. 11A-E illustrate views of a first ring according to another embodiment of the present invention. The first ring 204 is substantially identical in structure and operation to the first ring 104 of FIGS. 8A-C, except for the differences described below. The first ring 204 includes a slot 269, configured to receive at least one loop of the strap of the container. Thus, the slot prevents the strap of the container from slipping off the container, and the container is operable to be hung and supported solely by the strap. In one embodiment, the slot 269 is positioned below the top rim 270 of the first ring 204. In one embodiment, the first ring 204 includes a central ring body 226, a top flange 224, and a bottom flange 225, wherein the top flange 224 and the bottom flange 225 are at different ends of the first ring 204. In one embodiment, the top flange 224 includes a slot 269. In one embodiment, the first ring 204 further includes a threaded interior ring body 227.

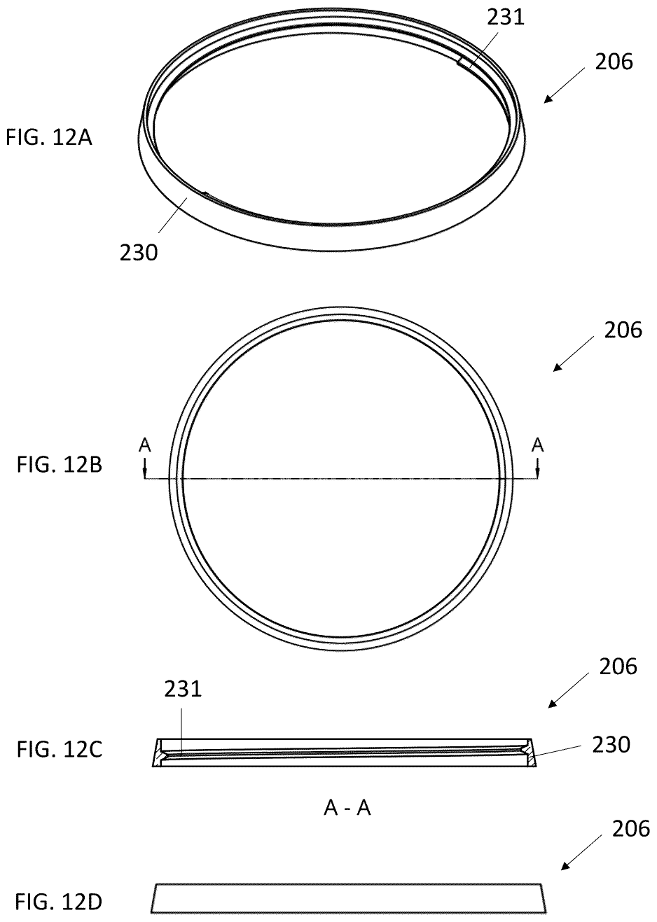

FIGS. 12A-D illustrate views of a second ring according to another embodiment of the present invention. The second ring 206 is substantially identical in structure and operation to the second ring 106 of FIGS. 9A-C and the third ring 111 of FIGS. 10A-C, except for the differences described below. Additionally, although the following description focuses on various embodiments of the second ring 206, the same modifications and variations are applicable to the third ring 211 in the present disclosure. In one embodiment, the second ring 206 includes a central ring body 230 and does not include one or more flanges. In one embodiment, the second ring 206 is tapered. In one embodiment, the second ring 206 further includes a threaded interior ring body 231.

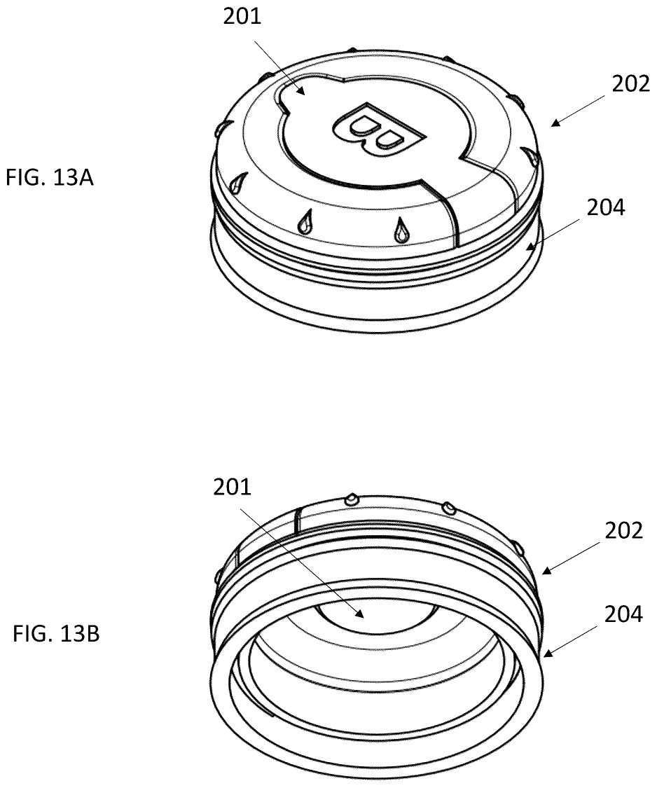

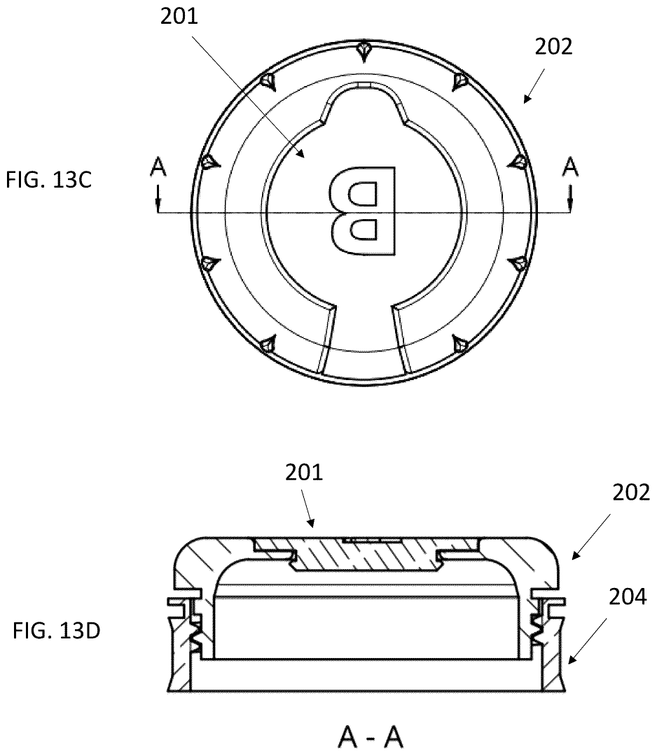

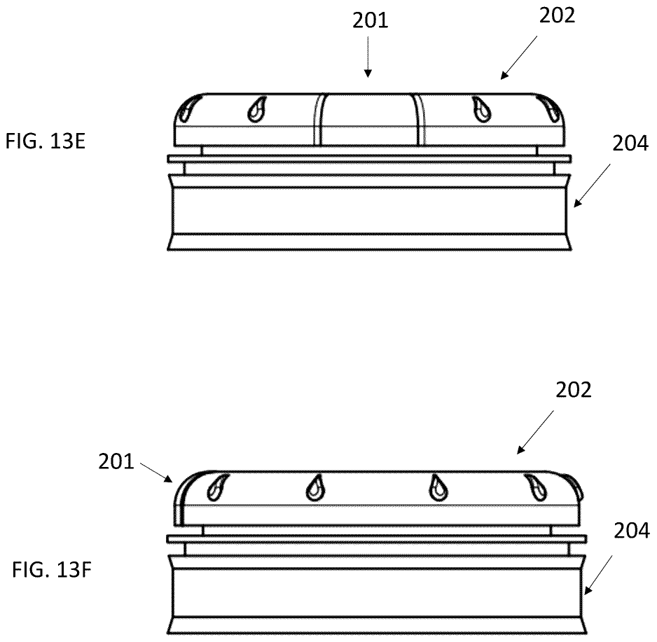

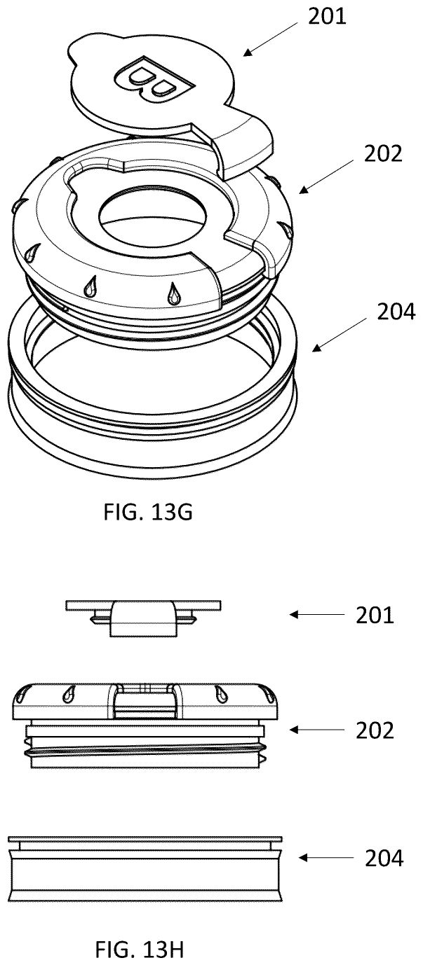

FIGS. 13A-H illustrate views of a lid, drainage cap, and ring assembly according to another embodiment of the present invention. The drainage cap 201 and the lid 202 are configured to fit together such that, when assembled, the assembly provides a watertight seal between the drainage cap 201 and the lid 202. This prevents fluid in the container from leaking out when the container is shaken and/or flipped upside down. The lid 202 is configured to connect to the superior body segment of the container via the ring 204. When the lid 202 and the ring 204 are assembled, loops of the strap of the container are operable to rest in the slot of the ring 204 and the slot formed above the collar of the lid 202. In one embodiment, the loops of the strap of the container are operable to rest in the slot of the ring 204.

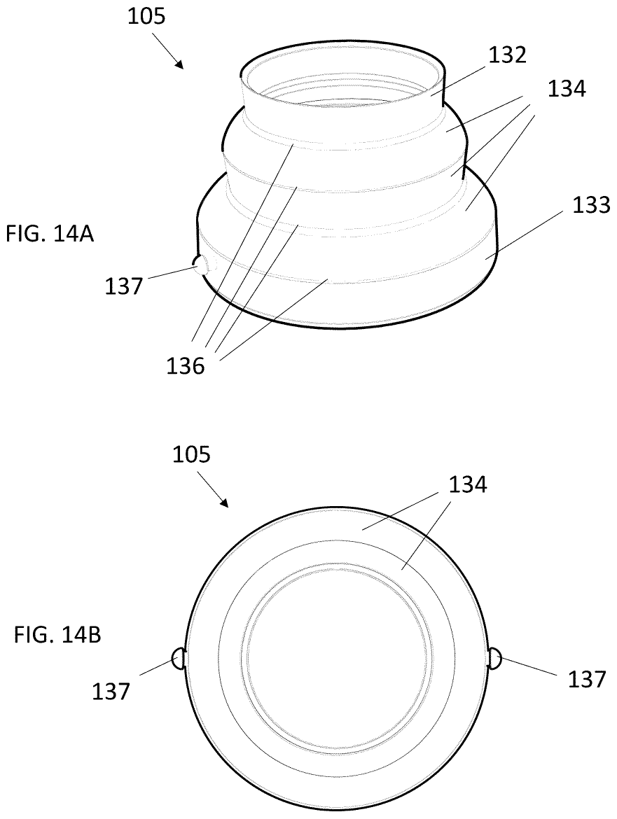

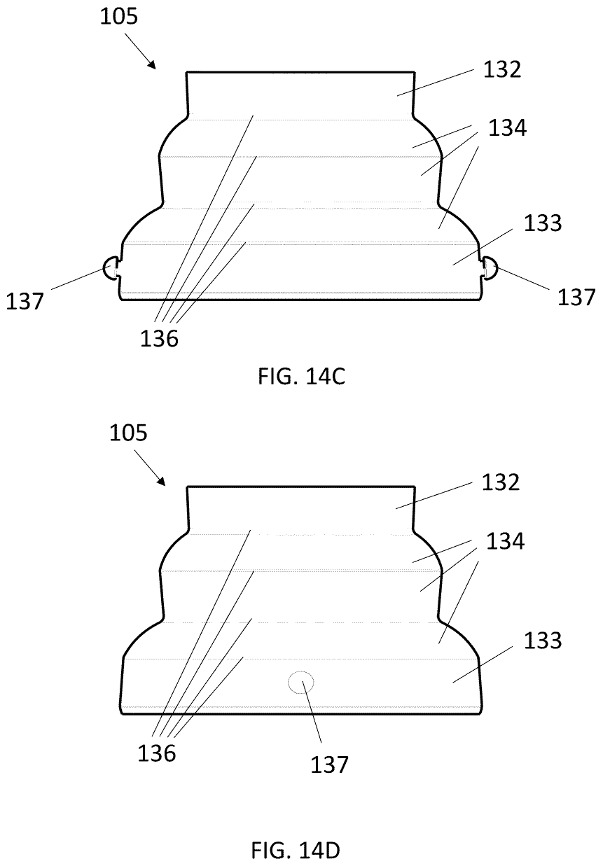

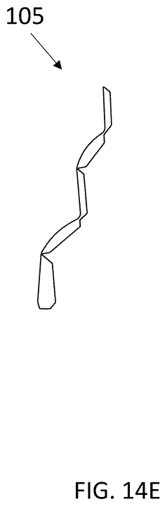

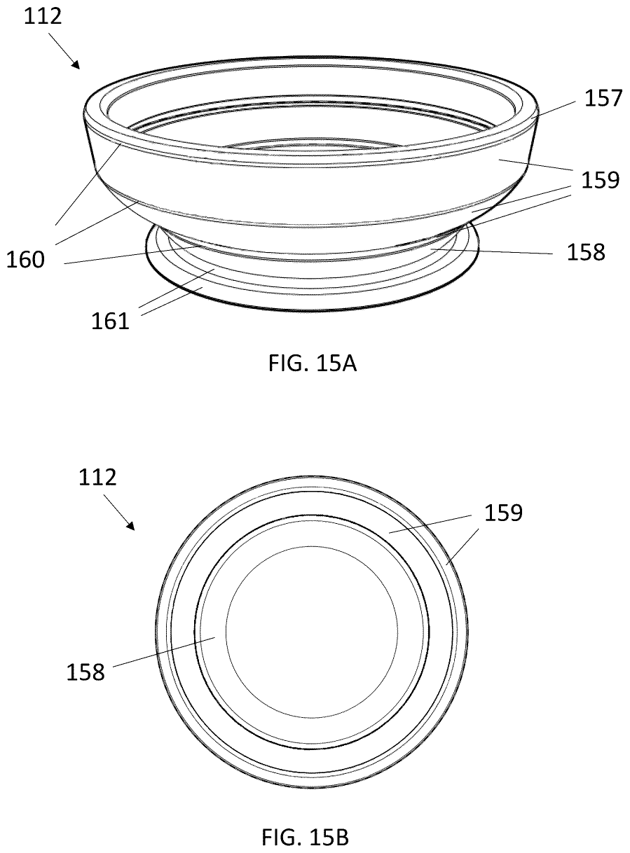

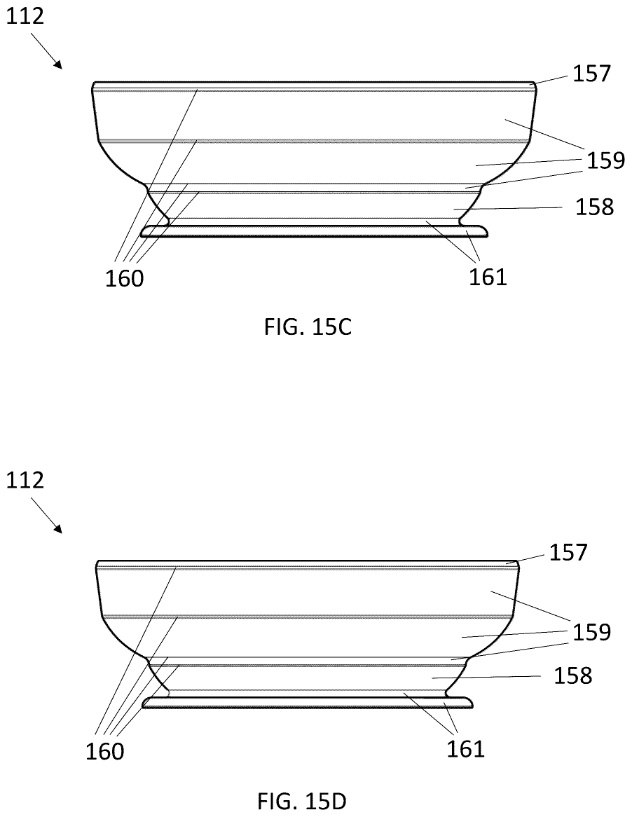

FIGS. 14A-15D illustrate views of a body segment. In one embodiment, the superior body segment 105 includes a plurality of flexible walls 132, 133, 134. In one embodiment, the superior body segment 105 further includes a plurality of crease lines 136, wherein the plurality of crease lines 136 form boundaries between adjacent flexible walls 132, 133, 134. In one embodiment, the superior body segment 105 is hollow. In one embodiment, the superior body segment 105 includes a top portion 132, a bottom portion 133, and the main body 134. In one embodiment, the superior body segment 105 is operable to store water used to clean the menstrual collection device.

In one embodiment, the superior body segment 105 includes varying radii at different heights, wherein a first radius at a first height is larger than the second radius at the second height, wherein the second height is taller than the first height. In one embodiment, the superior body segment 105 includes tiers of connecting flexible walls 132, 133, 134, wherein the diameter of a first tier is smaller than the diameter of the tier below it. In one embodiment, the outer diameter at the bottom portion 133 of the superior body segment 105 is approximately 95 mm, and the outer diameter at the top portion 132 of the superior body segment 105 is approximately 60 mm. In one embodiment, the height of the fully extended superior body segment 105 is approximately 60 mm.

In one embodiment, the superior body segment 105 further includes a plurality of body attachment posts 137. In one embodiment, the plurality of body attachment posts 137 are configured to receive a lanyard. One of ordinary skill in the art will appreciate the lanyard is operable to be substituted with any supportive feature (e.g., yarn, string, cord, webbing, handle) known in the art, wherein the container is operable to hang via the supportive feature. In one embodiment, the plurality of attachment posts 137 are spaced equidistantly apart. In one embodiment, the plurality of attachment posts 137 are operable to be removably attached to the superior body segment 105. In one embodiment, the superior body segment 105 including the plurality of attachment posts 137 form a unitary body.

In one embodiment, the inferior body segment 112 includes a plurality of flexible walls 157, 158, 159. In one embodiment, the inferior body segment 112 further includes a plurality of crease lines 160, wherein the plurality of crease lines 160 form boundaries between adjacent flexible walls 157, 158, 159. In one embodiment, the inferior body segment 112 includes one opening, wherein the inferior body segment 112 is operable to contain a reservoir of water. In one embodiment, the inferior body segment 112 includes a top portion 157, a bottom portion 158, and the main body 159.

In one embodiment, the inferior body segment 112 includes varying radii at different heights, wherein a first radius at a first height is smaller than the second radius at the second height, wherein the second height is taller than the first height. In one embodiment, the inferior body segment 112 includes tiers of connecting flexible walls 157, 158, 159, wherein the diameter of a first tier is larger than the diameter of the tier below it. In one embodiment, the outer diameter at the bottom portion 158 of the inferior body segment 112 is approximately 95 mm, and the outer diameter at the top portion 157 of the inferior body segment 112 is approximately 60 mm. In one embodiment, the height of the fully extended inferior body segment 112 is approximately 60 mm.

In one embodiment, the inferior body segment 112 includes a base 161. In one embodiment, the base 161 is connected to the bottom portion 133. In one embodiment, the container is operable to stand upright on the base 161. In one embodiment, the inferior body segment 112 is a unitary body.

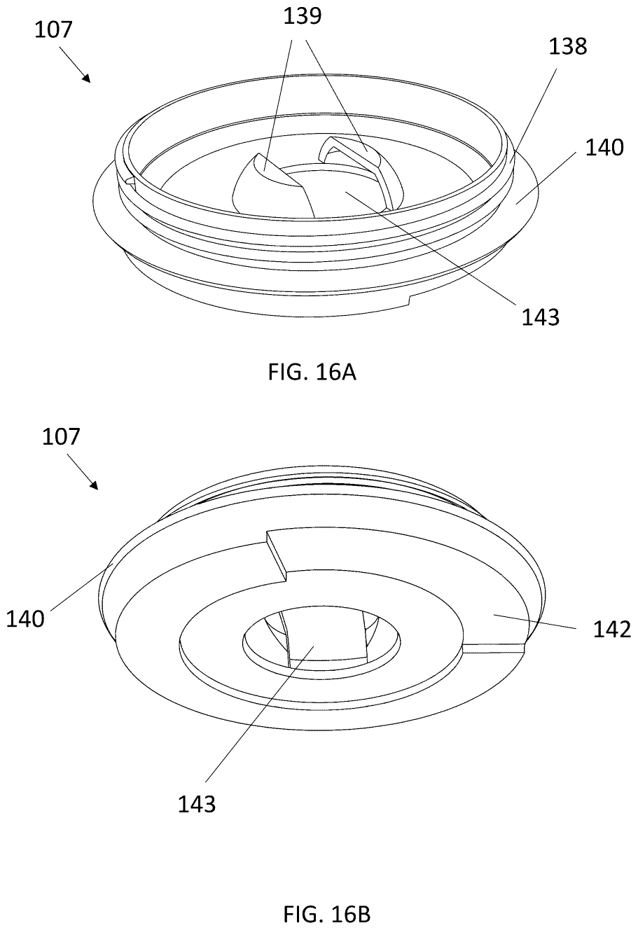

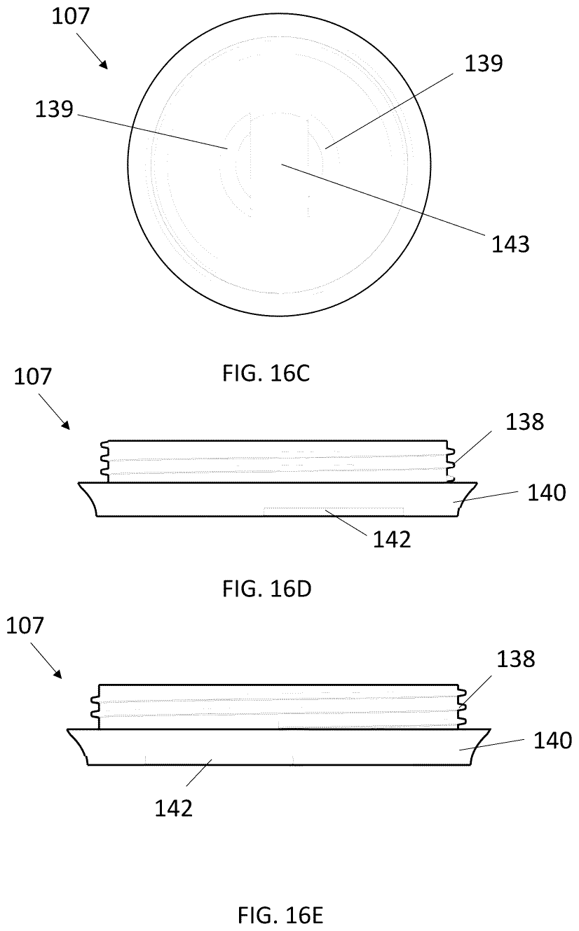

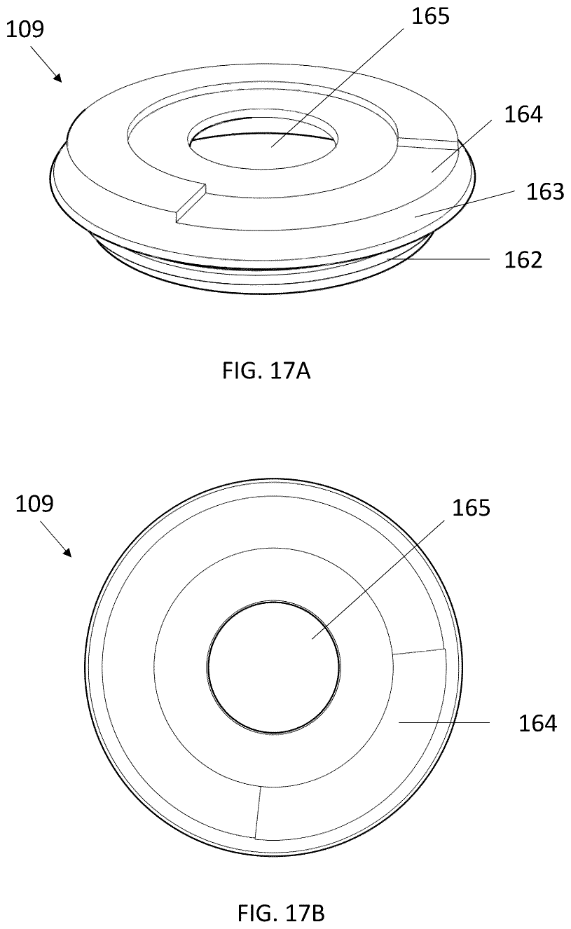

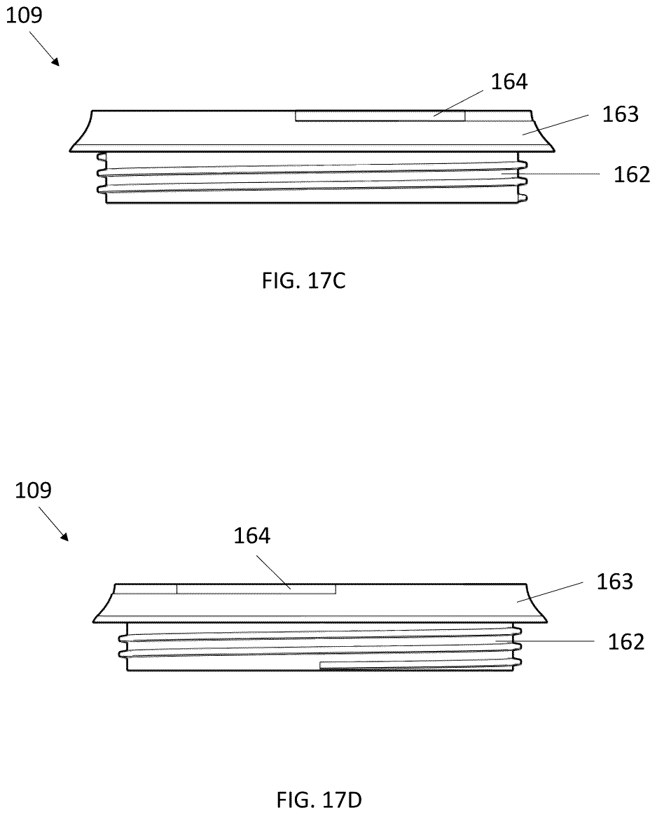

FIGS. 16A-17D illustrate views of a valve adapter according to one embodiment of the present invention. FIGS. 16A-E illustrate views of a superior valve adapter 107, and FIGS. 17A-D illustrate views of an inferior valve adapter 109. In one embodiment, the divider further includes at least one seal or rubber gasket, wherein the at least one seal or rubber gasket creates a waterproof seal. In one embodiment, the superior valve adapter 107 includes a threaded body 138, a plurality of occluding walls 139, wherein the plurality of occluding walls 139 partially surrounds an opening 143. The plurality of occluding walls 139 block fluid flow when the valve is turned into specific positions and allow fluid flow when the valve is turned into other positions. In one embodiment, the superior valve adapter 107 further includes a collar 140. In one embodiment, the collar 140 of the superior valve adapter 107 includes a recessed portion 142, wherein the recessed portion 142 provides the total degrees the valve is operable to turn. In one embodiment, the superior valve adapter 107 is configured to receive the valve, wherein the valve is operable to rotate independently from the superior valve adapter 107. In a preferred embodiment, the superior valve adapter 107 is configured to securely attach to the valve. In one embodiment, the valve includes a plurality of joining peg and the superior valve adapter 107 includes a plurality of corresponding interior holes. One of ordinary skill in the art will appreciate the valve and the valve adapters 107, 109 are operable to be joined together with suitable adhesive and/or other fasteners or fastening techniques so that a unitary assembly is formed.

In one embodiment, the maximum diameter of the collar 140 of the superior valve adapter 107 is approximately 95 mm. In one embodiment, the diameter of the opening 143 of the superior valve adapter 107 is approximately 33 mm. In one embodiment, the height of the superior valve adapter 107 is approximately 18 mm. In one embodiment, the height of the collar 140 of the superior valve adapter 107 is approximately 8 mm. In one embodiment, the height of the threaded body 138 of the superior valve adapter 107 is between approximately 10 mm and 14 mm. In one embodiment, the superior valve adapter 107 is configured to mate with the second ring and valve.

In one embodiment, the inferior valve adapter 109 includes a threaded body 162 and an opening 165. In one embodiment, the inferior valve adapter 109 includes a plurality of occluding walls, wherein the plurality of occluding walls partially surrounds an opening 165. The plurality of occluding walls block fluid flow when the valve is turned into specific positions and allow fluid flow when the valve is turned into other positions. In one embodiment, the inferior valve adapter 109 further includes a collar 163. In one embodiment, the collar 163 of the inferior valve adapter 109 includes a recessed portion 164, wherein the recessed portion 164 provides the total degrees the valve is operable to turn. In one embodiment, the inferior valve adapter 109 is configured to receive the valve, wherein the valve is operable to rotate independently from the inferior valve adapter 109. In a preferred embodiment, the inferior valve adapter 109 is configured to securely attach to the valve. In one embodiment, the valve includes a plurality of joining peg and the inferior valve adapter 109 includes a plurality of corresponding interior holes. One of ordinary skill in the art will appreciate the valve and the valve adapters 107, 109 are operable to be joined together with suitable adhesive and/or other fasteners or fastening techniques so that a unitary assembly is formed.

In one embodiment, the maximum diameter of the collar 163 of the inferior valve adapter 109 is approximately 95 mm. In one embodiment, the diameter of the opening 165 of the inferior valve adapter 109 is approximately 33 mm. In one embodiment, the height of the inferior valve adapter 109 is approximately 18 mm. In one embodiment, the height of the collar 163 of the inferior valve adapter 109 is approximately 8 mm. In one embodiment, the height of the threaded body 162 of the inferior valve adapter 109 is between approximately 10 mm and 14 mm. In one embodiment, the inferior valve adapter 109 is configured to mate with the third ring and valve.

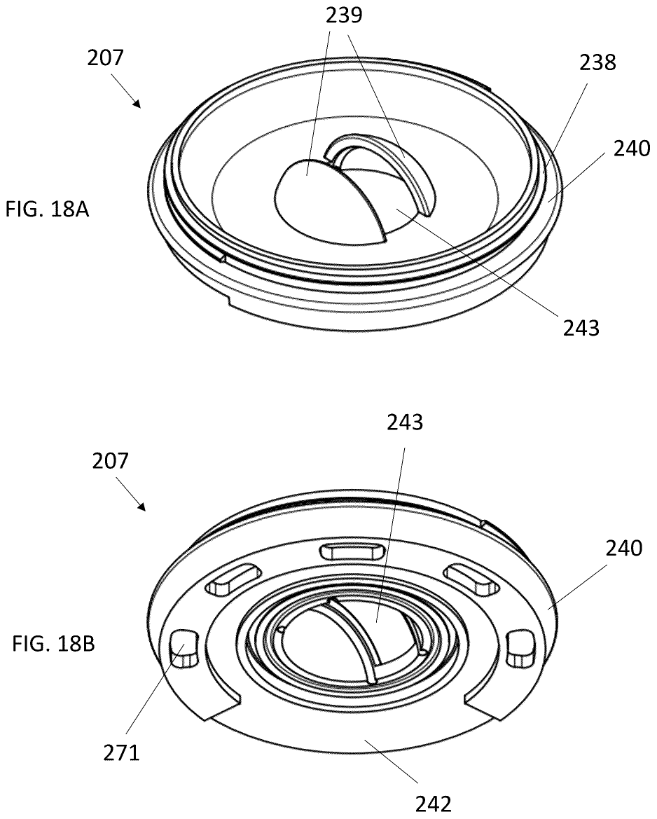

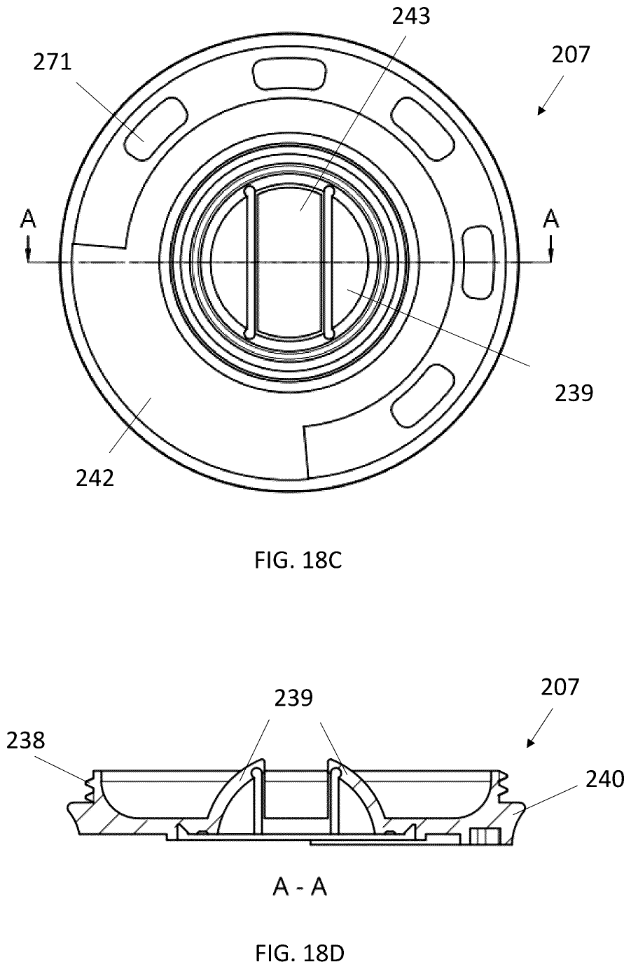

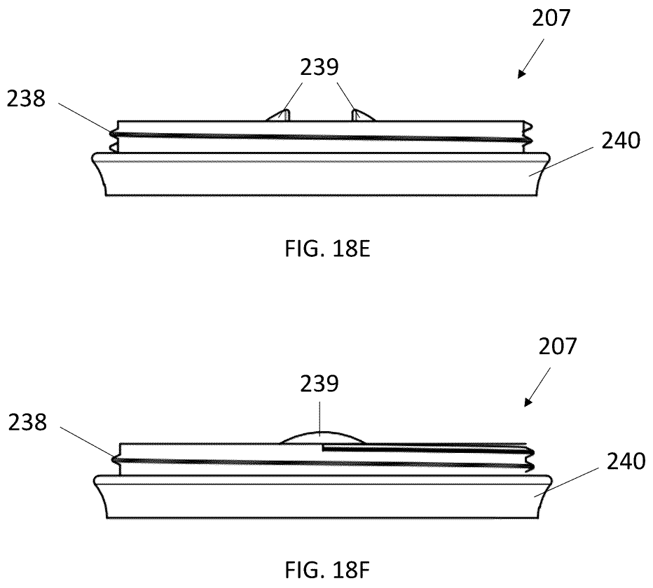

FIGS. 18A-F illustrate views of a superior valve adapter according to one embodiment of the present invention. The superior valve adapter 207 is substantially identical in structure and operation to the superior valve adapter 107 of FIGS. 16A-E, except for the differences that will now be described. The superior valve adapter 207 further includes a plurality of recesses 271, configured to mate with a corresponding plurality of locking protrusions of an inferior valve adapter. The locking protrusions and plurality of recesses of the inferior valve adapter and the superior valve adapter, respectively, increase the surface area and more securely “lock” the adapters together. This provides more stability to the assembly as the valve rotates independently of the valve adapters. In one embodiment, the superior valve adapter 207 includes a threaded body 238, a plurality of occluding walls 239, wherein the plurality of occluding walls 239 partially surrounds an opening 243. In one embodiment, the superior valve adapter 207 further includes a collar 240. In one embodiment, the collar 240 of the superior valve adapter 207 includes a recessed portion 242, wherein the recessed portion 242 provides the total degrees the valve is operable to turn.

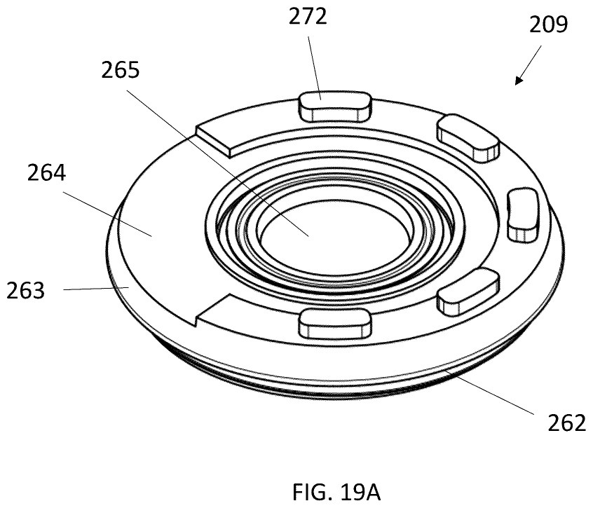

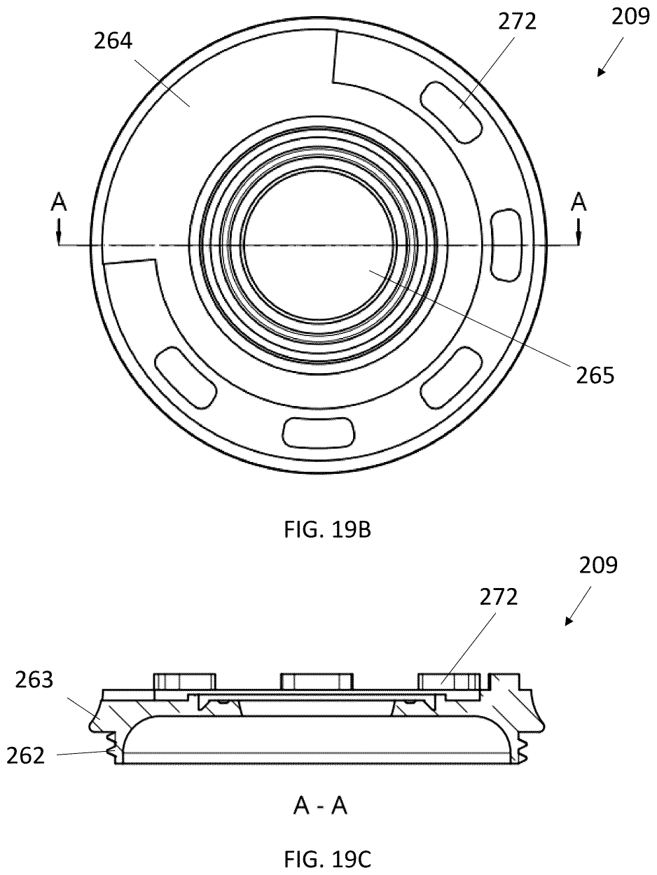

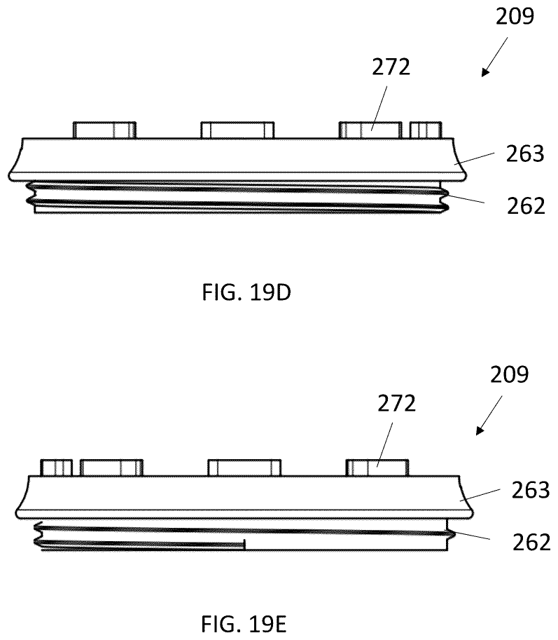

FIGS. 19A-E illustrate views of an inferior valve adapter according to one embodiment of the present invention. The inferior valve adapter 209 is substantially identical in structure and operation to the inferior valve adapter 109 of FIGS. 17A-D, except for the differences that will now be described. The inferior valve adapter 209 further includes a plurality of locking protrusions 272, configured to mate with a corresponding plurality of recesses of a superior valve adapter. The locking protrusions of the inferior valve adapter and the plurality of recesses of the superior valve adapter increase surface area and more securely “lock” the adapters together. This provides more stability to the assembly as the valve rotates independently of the valve adapters. In one embodiment, the inferior valve adapter 209 includes a threaded body 262 and an opening 265. In one embodiment, the inferior valve adapter 209 includes a plurality of occluding walls, wherein the plurality of occluding walls partially surrounds an opening 265. The plurality of occluding walls block fluid flow when the valve is turned into specific positions and allow fluid flow when the valve is turned into other positions. In one embodiment, the inferior valve adapter 209 further includes a collar 263. In one embodiment, the collar 263 of the inferior valve adapter 209 includes a recessed portion 264, wherein the recessed portion 264 provides the total degrees the valve is operable to turn.

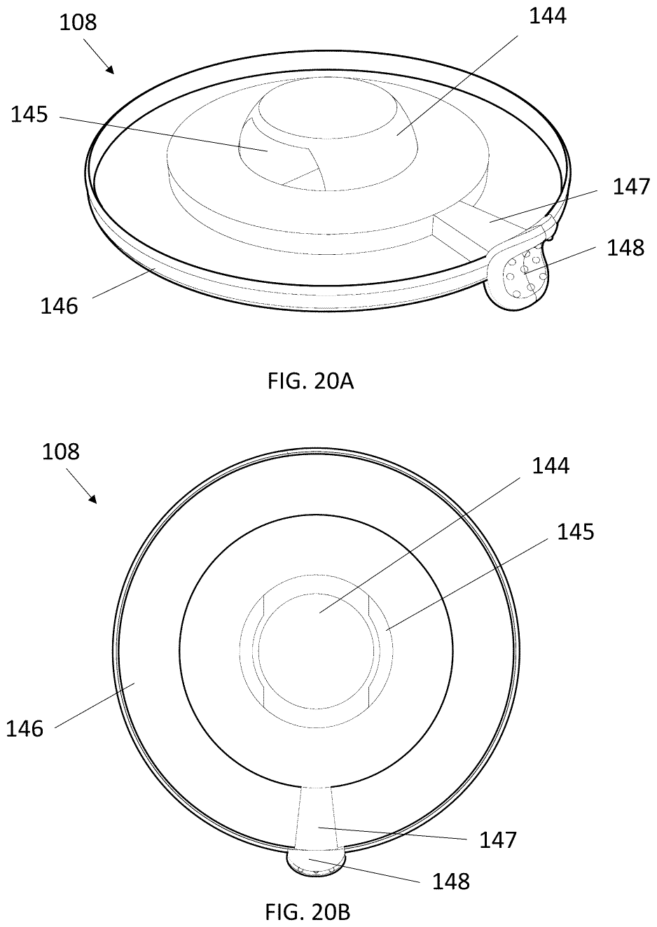

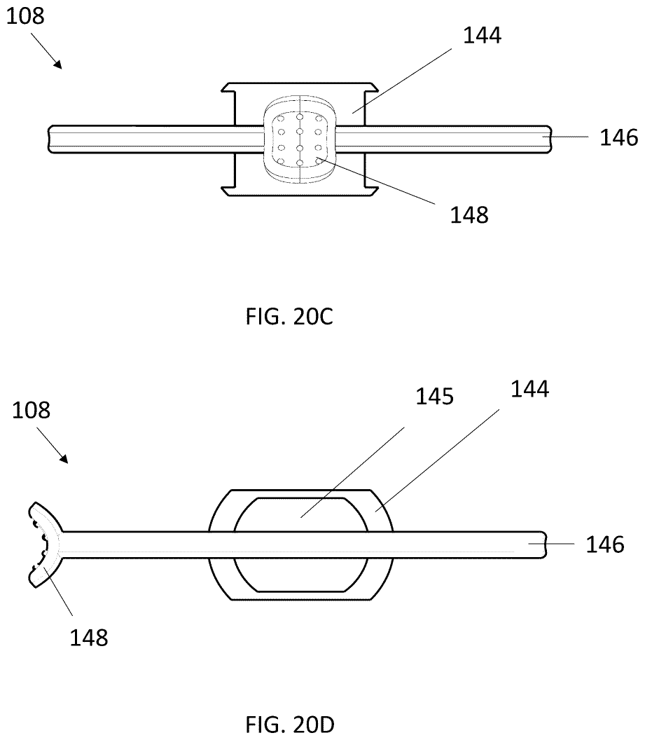

FIGS. 20A-D illustrate views of a valve according to one embodiment of the present invention. In one embodiment, the valve 108 includes a ball 144, a ball opening 145, and a lever 147. In one embodiment, the valve 108 further includes a handle 146, wherein the handle 146 forms a handwheel encircling the ball 144. One of ordinary skill in the art will appreciate that the ball 144 is analogous to that of a ball valve and thus, does not serve to limit the overall shape to solely a sphere. In one embodiment, the valve includes a plurality of ball openings 145, wherein the plurality of occluding walls of the valve adapter are configured to block fluid flow when the valve 108 is in a closed position.

In one embodiment, the start position of the valve 108 is the closed position. In one embodiment, the start position of the valve 108 is an open position, wherein fluid is free to flow throughout the container. In one embodiment, rotating the lever 147 counterclockwise around the valve 108 opens the valve, and rotating the lever 147 clockwise around the valve 108 closes the valve 108. In an alternative embodiment, rotating the lever 147 clockwise around the valve 108 opens the valve 108, and rotating the lever 147 counterclockwise around the valve 108 closes the valve 108. In one embodiment, the lever 147 connects the ball 144 to the handle 146. Thus, rotating the handle 146 opens or closes the valve 108 depending on the direction of the rotation.

In one embodiment, the handle 146 includes a textured pad 148. In one embodiment, the textured pad 148 is in line with the lever 147. In one embodiment, the textured pad 148 includes a plurality of raised elements and/or divots. Advantageously, the textured surface increases surface roughness to improve the user's grip when rotating the valve. In one embodiment, the valve 108 is a unitary body. In one embodiment, one or more textured surfaces are applied to the handle 146, wherein the one or more textured surfaces include a plurality of raised elements and/or divots.

In one embodiment, the maximum diameter of the valve 108 is approximately 90 mm. In one embodiment, the diameter of the handle 146 is approximately 90 mm. In one embodiment, the maximum width of the ball 144 is between approximately 28 mm to approximately 34 mm. In one embodiment, the height of the valve 108 is approximately 20 mm.

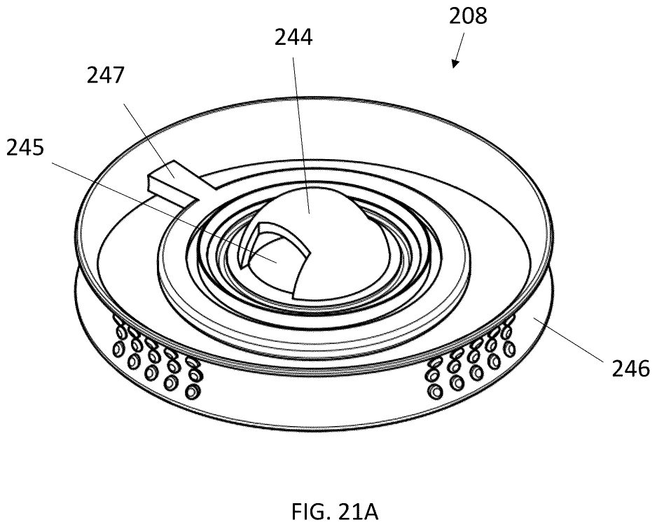

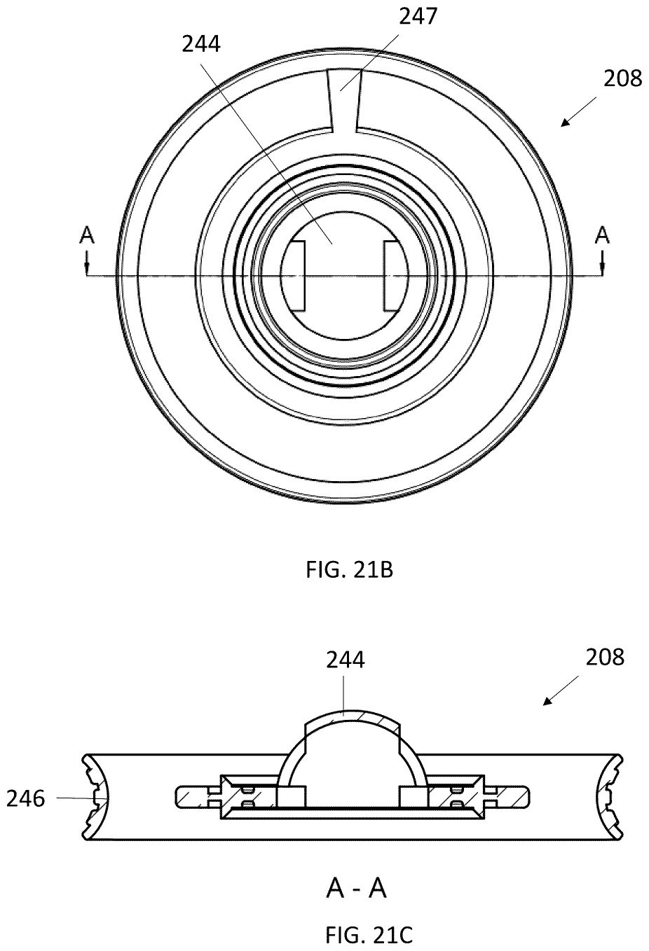

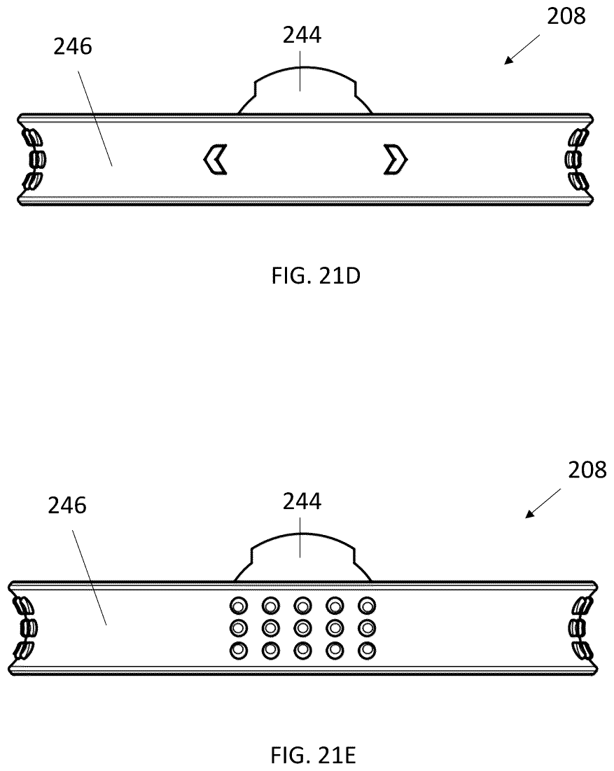

FIGS. 21A-E illustrate views of a valve according to another embodiment of the present invention. In one embodiment, the valve 208 includes a ball 244, a ball opening 245, and a lever 247. The valve 208 is substantially identical in structure and operation to the valve 108 of FIGS. 20A-D, except for the differences described below. In one embodiment, the handle 246 is shaped and sized to receive at least two fingers of a user's hand, promoting ergonomics when rotating the valve 208. In one embodiment, the handle 246 includes a plurality of textured surfaces spaced evenly or in a regular pattern around the handle 246. In another embodiment, the handle 246 includes a plurality of textured surfaces spaced randomly or in a semi-random pattern. In one embodiment, one or more textured surfaces include markings indicating the rotation direction for opening and/or closing the valve. In one embodiment, the handle 246 includes a logo or design.

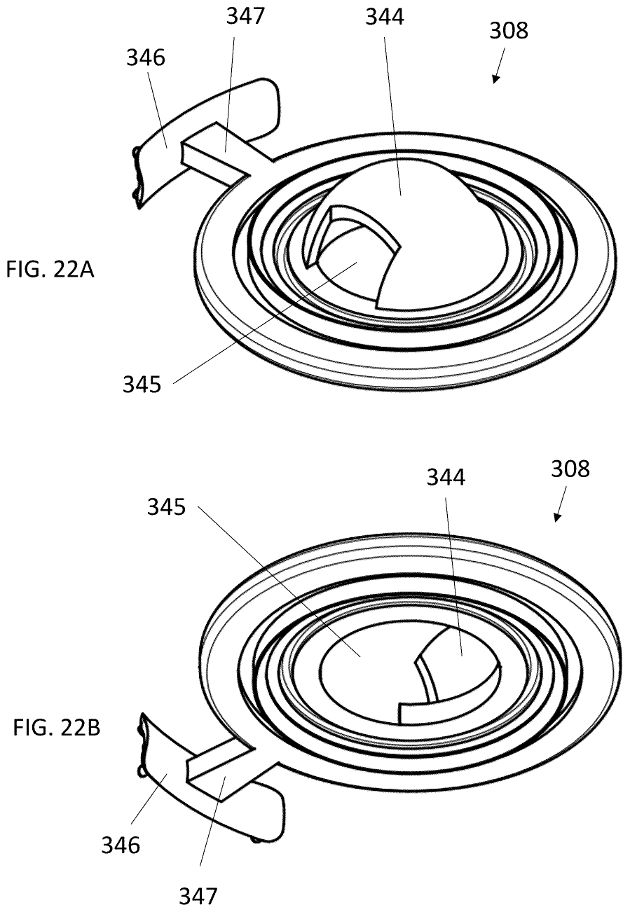

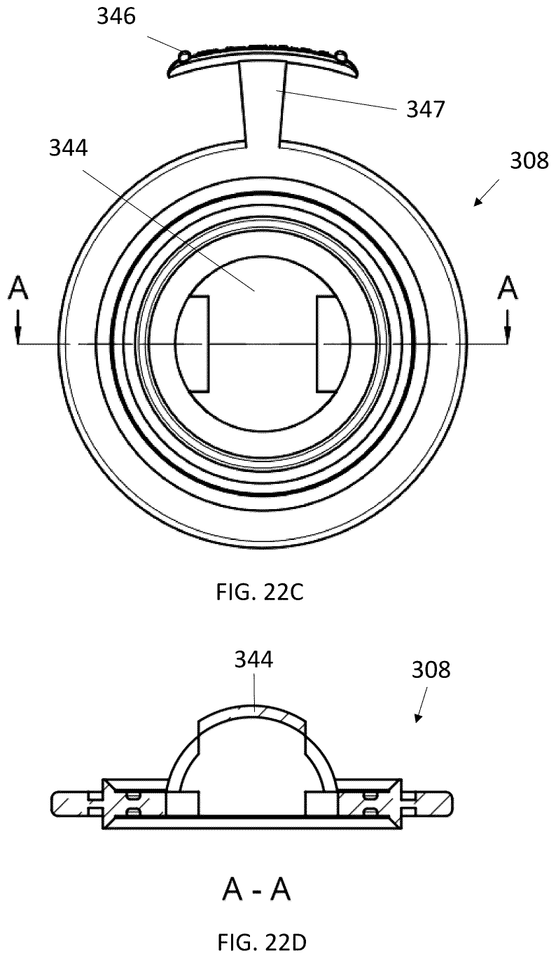

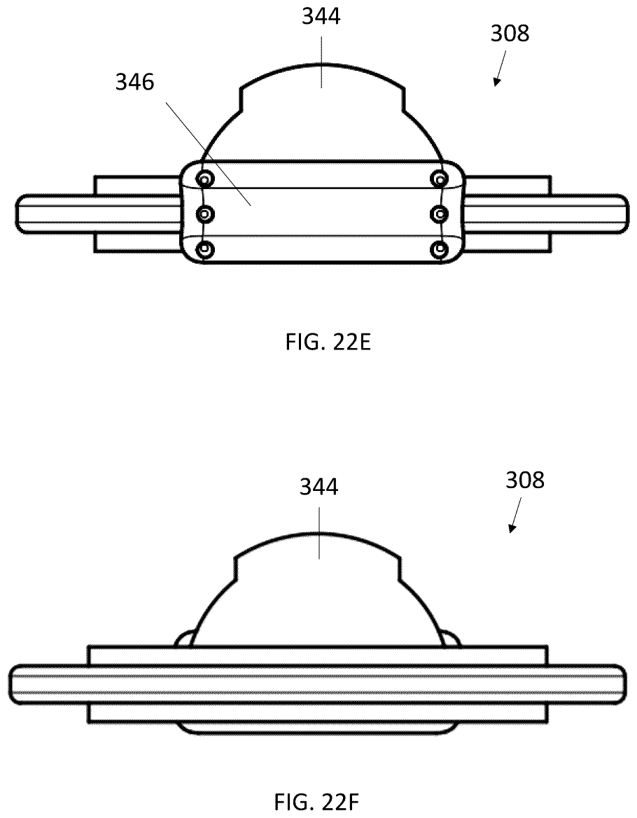

FIGS. 22A-F illustrate views of a valve according to yet another embodiment of the present invention. The valve 308 is substantially identical in structure and operation to the valve 108 of FIGS. 20A-D, except for the differences described below. In one embodiment, the handle 346 of the valve 308 does not encircle the ball 344. The valve 308 includes a ball opening 345 and a lever 347, wherein the lever 347 includes a handle 346. In one embodiment, the handle 346 is perpendicular to the lever 347. In one embodiment, the handle 346 includes a logo or design.

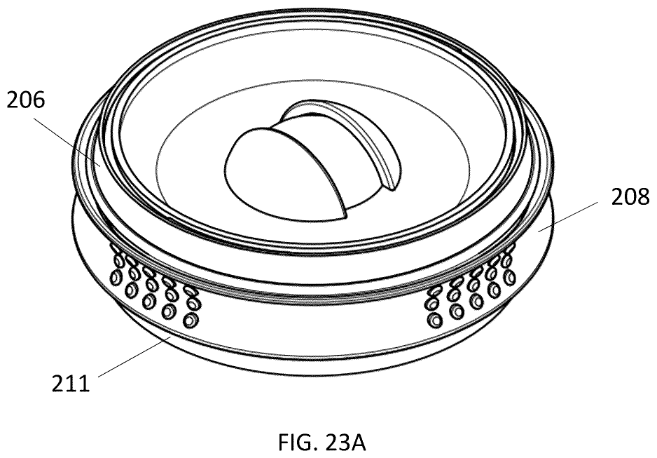

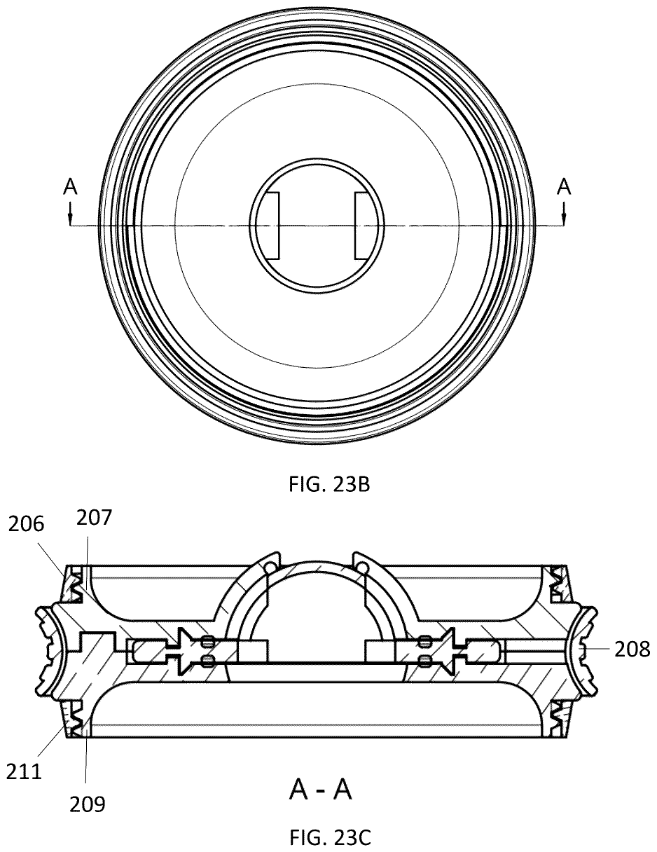

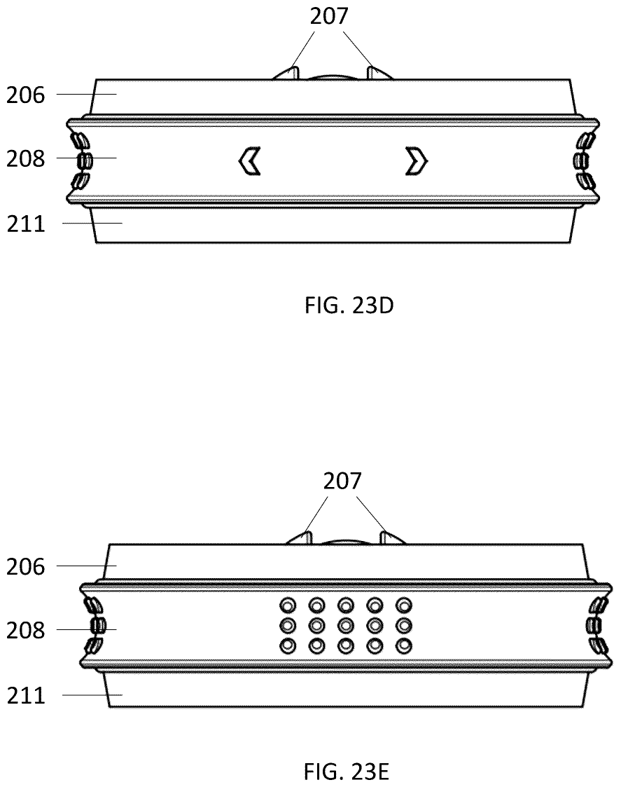

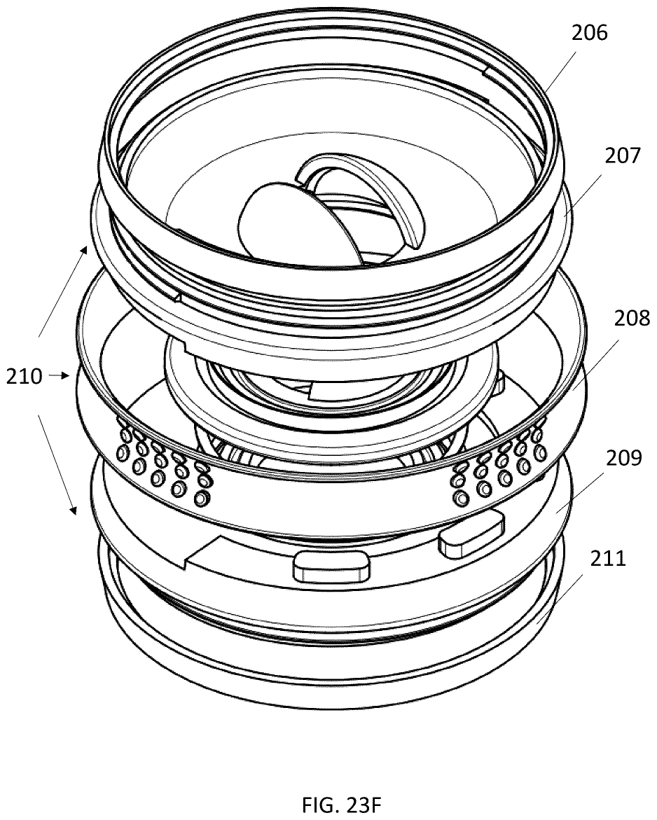

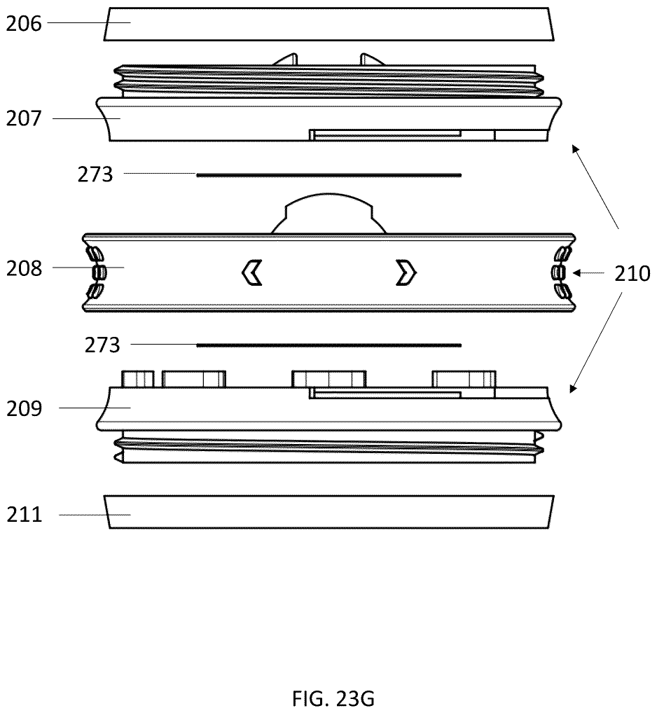

FIGS. 23A-G illustrate views of a divider and rings assembly according to another embodiment of the present invention. The divider 210 is substantially identical in structure and operation to the divider 110 of FIG. 2, except for the differences described below. In one embodiment, the divider 210 includes a valve 208 and two valve adapters 207, 209. In one embodiment, the divider 210 further includes at least one seal or rubber gasket 273, wherein the at least one seal or rubber gasket 273 creates a waterproof seal. The at least one seal or rubber gasket 273 is between the valve 208 and at least one valve adapter 207, 209.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

1. A container for cleaning a menstrual collection device, comprising:
a body including a superior body segment and an inferior body segment;
a divider including a valve and a plurality of valve adapters;
a lid; and
a strap;
wherein the superior body segment and the inferior body segment are connected via the divider;
wherein the superior body segment is connected to the lid via a threaded ring;
wherein the superior body segment and the inferior body segment comprise flexible walls, wherein the flexible walls are operable to reversely fold back, causing the container to collapse for storage;
wherein the lid includes a drainage port, and wherein the lid is configured to cover the drainage port with a drainage cap;
wherein the threaded ring includes a notch, operable to receive at least one loop of the strap;
wherein the valve is configured to rotate independently from the plurality of valve adapters; and
wherein the menstrual collection device is stored in the superior body segment during cleaning.

2. The container of claim 1, wherein the inferior body segment is operable to hold a reservoir of water for a second cleaning cycle.

3. The container of claim 1, wherein the valve includes a handle, wherein the handle includes one or more textured surfaces.

4. The container of claim 3, wherein the one or more textured surfaces include markings indicating a rotation direction for opening and/or closing the valve.

5. The container of claim 1, wherein the divider includes at least one seal or rubber gasket.

6. The container of claim 1, wherein the container is operable to fold closed in increments.

7. The container of claim 1, wherein the container is self-standing when containing a menstrual collection device.

8. The container of claim 1, wherein the body comprises silicone.

9. A container for cleaning a menstrual collection device, comprising:
a body including a superior body segment and an inferior body segment;
a divider including a valve and a plurality of valve adapters; and
a lid;
wherein the divider is configured to connect the superior body segment and the inferior body segment;
wherein the lid is configured to connect to the superior body segment;
wherein the body includes flexible walls;
wherein the container is operable to expand and collapse telescopically;
wherein the lid includes a drainage port and a drainage cap;
wherein the valve is configured to rotate independently from the plurality of valve adapters;
wherein the valve includes a handwheel encircling the valve;
wherein the inferior body segment is operable to hold a reservoir of water; and
wherein the superior body segment is configured to store the menstrual collection device.

10. The container of claim 9, wherein the drainage cap is removably attached to the lid.

11. The container of claim 9, wherein the container is self-standing when the reservoir is filled with water.

12. The container of claim 9, wherein the container is self-standing when the reservoir is partially filled with water.

13. The container of claim 9, further including a strap operable to support the container when hung by the strap.

14. The container of claim 9, further comprising an ultraviolet (UV) module configured to sanitize and/or disinfect the menstrual collection device.

15. The container of claim 9, wherein the body comprises silicone.

16. A container for cleaning a menstrual collection device, comprising:
a superior body segment;
an inferior body segment;
a valve;
a superior valve adapter;
an inferior valve adapter; and
a lid;
wherein the lid is connected to the superior body segment via a threaded ring;
wherein the superior body segment is connected to the valve via the superior valve adapter;
wherein the inferior body segment is connected to the valve via the inferior body adapter;
wherein both the superior body segment and the inferior body segment include flexible walls;
wherein a force applied to at least one end of the container causes the flexible walls to reversibly fold back to an inverted position;
wherein the lid includes a drainage port;
wherein the valve is configured to rotate independently from the superior valve adapter;
wherein the inferior body segment is operable to hold a reservoir of water; and
wherein the superior body segment is configured to contain the menstrual collection device during cleaning.

17. The container of claim 16, further including a strap.

18. The container of claim 16, wherein the superior valve adapter includes a plurality of occluding walls, wherein the plurality of occluding walls block fluid flow when the valve is at a first position and allow fluid flow when the valve is at a second position.

19. The container of claim 16, wherein the drainage port is operable to be covered by a drainage cap.

20. The container of claim 16, wherein the superior body segment and the inferior body segment comprise silicone.
